SEQUENCE LISTING (80 sequences)

SEQ ID NO: 1 - protein, 64 aa, Trichophyton rubrum
LISVINILAQALYSIFAAGDPNLKVLQRGCFRYFQLGLIDGPIGLLSGIIRSPLVLLRHFYSVA

SEQ ID NO: 2 - protein, 64 aa, Trichophyton interdigitale
LISVINILAQSLYSIFAAGDPNLKVLQRGCFRYFQLGLIDGPIGLLSGIIRSPLVLLRHFYSVA

SEQ ID NO: 3 - protein, 64 aa, Trichophyton mentagrophytes; identical to SEQ ID NO: 2

SEQ ID NO: 4 - protein, 64 aa, Aspergillus niger
ASSVINILAQALYALFAANDENLRALQRGCFRYFELGMTSGPIGLLGGLIKKPDVLFWHFFSVA

SEQ ID NO: 5 - DNA, 65 bp, synthetic construct
  source 1..65: mol_type = other DNA
ggtgacagattagtaatccgccaagcgcgcatcaggcttgcaagtctccggcgaatatagagtaa

SEQ ID NO: 6 - DNA, 26 bp, synthetic construct
  source 1..26: mol_type = other DNA
cttgcaagtctccggcgaatatagag

SEQ ID NO: 7 - DNA, 62 bp, synthetic construct
  source 1..62: mol_type = other DNA
cgtgattcatgccgaccttatatgaccgacagactgaccgagcttgcattaccagcggcgag

SEQ ID NO: 8 - DNA, 21 bp, synthetic construct
  source 1..21: mol_type = other DNA
cgagcttgcattaccagcggc

SEQ ID NO: 9 - DNA, 68 bp, synthetic construct
  source 1..68: mol_type = other DNA
ggtgacagattagtaatccgccaagcgcgcatcaggtgtaagcagtgagtctataagtccaagttggg

SEQ ID NO: 10 - DNA, 31 bp, synthetic construct
  source 1..31: mol_type = other DNA
aggtgtaagcagtgagtctataagtccaagt

SEQ ID NO: 11 - DNA, 23 bp, synthetic construct
  source 1..23: mol_type = other DNA
gacagattagtaatccgccaagc

SEQ ID NO: 12 - DNA, 24 bp, synthetic construct
  source 1..24: mol_type = other DNA
gattcatgccgaccttatatgacc

SEQ ID NO: 13 - DNA, 20 bp, synthetic construct
  source 1..20: mol_type = other DNA
ttccactggcaacggaagtc

SEQ ID NO: 14 - DNA, 22 bp, synthetic construct
  source 1..22: mol_type = other DNA
cgacgccaacgataagatcctg

SEQ ID NO: 15 - DNA, 19 bp, synthetic construct
  source 1..19: mol_type = other DNA
ggcactggactccaaggac

SEQ ID NO: 16 - DNA, 22 bp, synthetic construct
  source 1..22: mol_type = other DNA
tcaccattgtcgagaccaaggc

SEQ ID NO: 17 - DNA, 68 bp, synthetic construct
  source 1..68: mol_type = other DNA
gctttccgagttgtgcaatccagatctcgttggctccgcttgcaagtctccggcgaatatagagtatg

SEQ ID NO: 18 - DNA, 26 bp, synthetic construct
  source 1..26: mol_type = other DNA
cttgcaagtctccggcgaatatagag

SEQ ID NO: 19 - DNA, 62 bp, synthetic construct
  source 1..62: mol_type = other DNA
acgtactgattggcaaccggagtgtattgcagatgcagaatatgttgagtccaccagcggct

SEQ ID NO: 20 - DNA, 26 bp, synthetic construct
  source 1..26: mol_type = other DNA
atgcagaatatgttgagtccaccagc

SEQ ID NO: 21 - DNA, 69 bp, synthetic construct
  source 1..69: mol_type = other DNA
acagtgacgcagaggctcttggtctttcagctcagacgacaacgagtaatctataagtccaagttggat

SEQ ID NO: 22 - DNA, 33 bp, synthetic construct
  source 1..33: mol_type = other DNA
agacgacaacgagtaatctataagtccaagttg

SEQ ID NO: 23 - DNA, 22 bp, synthetic construct
  source 1..22: mol_type = other DNA
ccgagttgtgcaatccagatct

SEQ ID NO: 24 - DNA, 20 bp, synthetic construct
  source 1..20: mol_type = other DNA
actgattggcaaccggagtg

SEQ ID NO: 25 - DNA, 19 bp, synthetic construct
  source 1..19: mol_type = other DNA
gacgcagaggctcttggtc

SEQ ID NO: 26 - DNA, 20 bp, synthetic construct
  source 1..20: mol_type = other DNA
ttccactggcaacggaagtc

SEQ ID NO: 27 - DNA, 22 bp, synthetic construct
  source 1..22: mol_type = other DNA
cgacgccaacgataagatcctg

SEQ ID NO: 28 - DNA, 19 bp, synthetic construct
  source 1..19: mol_type = other DNA
ggcactggactccaaggac

SEQ ID NO: 29 - DNA, 22 bp, synthetic construct
  source 1..22: mol_type = other DNA
tcaccattgtcgagaccaaggc

SEQ ID NO: 30 - DNA, 66 bp, synthetic construct
  source 1..66: mol_type = other DNA
acgtactgattggcaaccggagtgtattgcagtgtaagcagtgagtccagkccttatactctataa

SEQ ID NO: 31 - DNA, 71 bp, synthetic construct
  source 1..71: mol_type = other DNA
atcatgtagtacgcgattcaagctctctgacagagtctgtaagcagtgagtccagkccttatactctatag

SEQ ID NO: 32 - DNA, 28 bp, synthetic construct
  source 1..28: mol_type = other DNA
tgtaagcagtgagtccagkccttatact

SEQ ID NO: 33 - DNA, 65 bp, synthetic construct
  source 1..65: mol_type = other DNA
acagtgacgcagaggctcttggtctttcagctcagacgacaacgagtaatgctgcttcaggtatg

SEQ ID NO: 34 - DNA, 26 bp, synthetic construct
  source 1..26: mol_type = other DNA
agacgacaacgagtaatgctgcttca

SEQ ID NO: 35 - DNA, 68 bp, synthetic construct
  source 1..68: mol_type = other DNA
gctttccgagttgtgcaatccagatctcgttggctccgcttgcaagtctcccttggttctattacgrt

SEQ ID NO: 36 - DNA, 28 bp, synthetic construct
  source 1..28: mol_type = other DNA
cgcttgcaagtctcccttggttctatta

SEQ ID NO: 37 - DNA, 22 bp, synthetic construct
  source 1..22: mol_type = other DNA
ccgagttgtgcaatccagatct

SEQ ID NO: 38 - DNA, 20 bp, synthetic construct
  source 1..20: mol_type = other DNA
actgattggcaaccggagtg

SEQ ID NO: 39 - DNA, 19 bp, synthetic construct
  source 1..19: mol_type = other DNA
gacgcagaggctcttggtc

SEQ ID NO: 40 - DNA, 23 bp, synthetic construct
  source 1..23: mol_type = other DNA
atgtagtacgcgattcaagctct

SEQ ID NO: 41 - DNA, 25 bp, synthetic construct
  source 1..25: mol_type = other DNA
agatgggtttgctagtaaggtgtag

SEQ ID NO: 42 - DNA, 21 bp, synthetic construct
  source 1..21: mol_type = other DNA
ccttgaccctttycgagtgca

SEQ ID NO: 43 - DNA, 23 bp, synthetic construct
  source 1..23: mol_type = other DNA
tgtctagctttgaagttcggcaa

SEQ ID NO: 44 - DNA, 23 bp, synthetic construct
  source 1..23: mol_type = other DNA
cctgcagggtcatccttcctttc

SEQ ID NO: 45 - DNA, 65 bp, synthetic construct
  source 1..65: mol_type = other DNA
gctttccgagttgtgcaatccagatctcgttggctcttgcaagtctcgcggcgaatatagagtag

SEQ ID NO: 46 - DNA, 26 bp, synthetic construct
  source 1..26: mol_type = other DNA
cttgcaagtctcgcggcgaatataga

SEQ ID NO: 47 - DNA, 59 bp, synthetic construct
  source 1..59: mol_type = other DNA
acagtgacgcagaggctcttggtctttcagctcacagaacgagtaatccaccagcggcc

SEQ ID NO: 48 - DNA, 24 bp, synthetic construct
  source 1..24: mol_type = other DNA
cacagaacgagtaatccaccagcg

SEQ ID NO: 49 - DNA, 22 bp, synthetic construct
  source 1..22: mol_type = other DNA
ccgagttgtgcaatccagatct

SEQ ID NO: 50 - DNA, 20 bp, synthetic construct
  source 1..20: mol_type = other DNA
actgattggcaaccggagtg

SEQ ID NO: 51 - DNA, 28 bp, synthetic construct
  source 1..28: mol_type = other DNA
cgttgatttctgtcatcaatattcttgc

SEQ ID NO: 52 - DNA, 22 bp, synthetic construct
  source 1..22: mol_type = other DNA
cgacgccaacgataagatcctg

SEQ ID NO: 53 - DNA, 19 bp, synthetic construct
  source 1..19: mol_type = other DNA
ggcactggactccaaggac

SEQ ID NO: 54 - DNA, 22 bp, synthetic construct
  source 1..22: mol_type = other DNA
tcaccattgtcgagaccaaggc

SEQ ID NO: 55 - DNA, 28 bp, synthetic construct
  source 1..28: mol_type = other DNA
catcaatattcttgctcaggccttatac

SEQ ID NO: 56 - DNA, 22 bp, synthetic construct
  source 1..22: mol_type = other DNA
tgcaggactttcaaattrgggt

SEQ ID NO: 57 - DNA, 28 bp, synthetic construct
  source 1..28: mol_type = other DNA
tcttgctcaggccttatactctatattc

SEQ ID NO: 58 - DNA, 22 bp, synthetic construct
  source 1..22: mol_type = other DNA
tgcaggactttcaaattrgggt

SEQ ID NO: 59 - DNA, 22 bp, synthetic construct
  source 1..22: mol_type = other DNA
acgaggctgcttcaggtatttc

SEQ ID NO: 60 - DNA, 23 bp, synthetic construct
  source 1..23: mol_type = other DNA
tgtctagctttgaagttcggcaa

SEQ ID NO: 61 - DNA, 22 bp, synthetic construct
  source 1..22: mol_type = other DNA
tcacccttggttctattacggc

SEQ ID NO: 62 - DNA, 23 bp, synthetic construct
  source 1..23: mol_type = other DNA
tgtctagctttgaagttcggcaa

SEQ ID NO: 63 - DNA, 24 bp, synthetic construct
  source 1..24: mol_type = other DNA
atcaatattcttgcycagkcctty

SEQ ID NO: 64 - DNA, 22 bp, synthetic construct
  source 1..22: mol_type = other DNA
tgcaggactttcaaattrgggt

SEQ ID NO: 65 - DNA, 23 bp, synthetic construct
  source 1..23: mol_type = other DNA
atcaatattcttgctcaggcctc

SEQ ID NO: 66 - DNA, 22 bp, synthetic construct
  source 1..22: mol_type = other DNA
tgcaggactttcaaattrgggt

SEQ ID NO: 67 - DNA, 27 bp, synthetic construct
  source 1..27: mol_type = other DNA
cttgctcaggccttatactctatattr

SEQ ID NO: 68 - DNA, 22 bp, synthetic construct
  source 1..22: mol_type = other DNA
tgcaggactttcaaattrgggt

SEQ ID NO: 69 - DNA, 25 bp, synthetic construct
  source 1..25: mol_type = other DNA
cttgctcaggccttatactctatav

SEQ ID NO: 70 - DNA, 22 bp, synthetic construct
  source 1..22: mol_type = other DNA
tgcaggactttcaaattrgggt

SEQ ID NO: 71 - DNA, 20 bp, synthetic construct
  source 1..20: mol_type = other DNA
acgaggctgcttcaggtatr

SEQ ID NO: 72 - DNA, 25 bp, synthetic construct
  source 1..25: mol_type = other DNA
agatgggtttgctagtaaggtgtag

SEQ ID NO: 73 - DNA, 20 bp, synthetic construct
  source 1..20: mol_type = other DNA
cgaggctgcttcaggtattc

SEQ ID NO: 74 - DNA, 25 bp, synthetic construct
  source 1..25: mol_type = other DNA
agatgggtttgctagtaaggtgtag

SEQ ID NO: 75 - DNA, 21 bp, synthetic construct
  source 1..21: mol_type = other DNA
cacccttggttctattacgrt

SEQ ID NO: 76 - DNA, 23 bp, synthetic construct
  source 1..23: mol_type = other DNA
tgtctagctttgaagttcggcaa

SEQ ID NO: 77 - DNA, 20 bp, synthetic construct
  source 1..20: mol_type = other DNA
ttccactggcaacggaagtc

SEQ ID NO: 78 - DNA, 25 bp, synthetic construct
  source 1..25: mol_type = other DNA
agatgggtttgctagtaaggtgtag

SEQ ID NO: 79 - DNA, 21 bp, synthetic construct
  source 1..21: mol_type = other DNA
atggttgtagaggctcctccc

SEQ ID NO: 80 - DNA, 27 bp, synthetic construct
  source 1..27: mol_type = other DNA
ctagctttgaagttcggcaaataygaa

(19) United States
(12) Patent Application Publication
(10) Pub. No.: US 2023/0323432 A1
(43) Pub. Date: Oct. 12, 2023

(54) ASSAY FOR DETECTING POINT MUTATIONS

(71) Applicant: Bakotic Pathology Associates, LLC, Alpharetta, GA (US)

(72) Inventors: Kiran Madanahally Divakar, Alpharetta, GA (US); Liquan Yang, Lilburn, GA (US); Rama Murthy Sakamuri, Cumming, GA (US); Laurence McCarthy, Alpharetta, GA (US)

(21) Appl. No.: 18/186,143

(22) Filed: Mar. 17, 2023

Related U.S. Application Data
(60) Provisional application No. 63/329,786, filed on Apr. 11, 2022.

(51) Int. Cl. C12Q 1/6827

(52) U.S. Cl. CPC C12Q 1/6827

(57) ABSTRACT

A method of sample analysis is provided. In some embodiments the method may comprise: hybridizing a tailed primer to a sample that comprises both wild type copies of a genomic locus and mutant copies of the genomic locus, extending the tailed primer using the genomic locus as a template to produce a primer extension product and detecting the primer extension product in a qPCR assay that employs a forward primer that is complementary to a sequence in the complement of the 5′ tail of the tailed primer, a reverse primer, and a hydrolysis probe that is complementary to at least 6 nucleotides of the target complementary sequence of the tailed primer and at least 6 nucleotides of the 5′ tail of the tailed primer. Kits for practice the method are also provided.

### CROSS-REFERENCE TO RELATED APPLICATIONS

Pursuant to 35 U.S.C. § 119 (e), this application claims priority to the filing date of U.S. Provisional Patent Application Ser. No. 63/329,786 filed Apr. 11, 2022, the disclosure of which application is herein incorporated by reference.

### INCORPORATION BY REFERENCE OF SEQUENCE LISTING PROVIDED AS A SEQUENCE LISTING XML FILE

A Sequence Listing is provided herewith as a Sequence Listing XML, “BAKO-003_SEQ_LIST” created Mar. 17, 2023 and having a size of 72,173 bytes. The contents of the Sequence Listing XML are incorporated by reference herein in their entirety.

### BACKGROUND

Onychodystrophy is any alteration of nail morphology and encompasses various pathological processes including infectious agents such as fungus and bacteria, non-infectious inflammatory dermatological diseases and tumors. Of the infectious causes the most common is by infection with fungal agents (onychomycosis). The main onychomycosis causative agents include dermatophyte, saprophyte, and yeasts. The most common pathogens implicated in onychomycosis are Trichophyton rubrum and Trichophyton mentagrophytes dermatophytic fungi. Accurate identification of the pathogenic fungi is beneficial when making decisions associated with anti-fungal therapy as not all antifungal agents are effective against every fungus, and different treatment regimens may be prescribed for different class or organisms. Currently terbinafine is widely used for the treatment of dermatophytic fungi. Two new drugs, efinaconazole (Jublia®) and tavaborole (Kerydin®), are indicated for the treatment of T. rubrum and T. mentagrophytes and may require a positive diagnosis for one of these specific organisms for treatment covered by a patient's insurer. As mutation-driven drug resistance toward terbinafine causing ineffective treatment has been reported in various literature worldwide, a mutation analysis test would assist the physicians to understand the potential risk of ineffectiveness of using terbinafine.

One of the driving factors for terbinafine resistance are point mutations in the Squalene epoxidase gene, which is involved ergosterol biosynthesis of Trychophyton sps, the target for terbinafine. Squalene epoxidase is involved in early stage of ergosterol biosynthesis, Inhibition of squalene epoxidase enzyme activity results in accumulation of Squalene, which is toxic to the fungi. There is an ongoing need for reliable and sensitive methods for detecting point mutations, particularly for the analysis of clinical samples that contain an excess of DNA molecules that do not contain the point mutation.

### SUMMARY

A method of sample analysis is provided. In some embodiments the method may comprise: hybridizing a tailed primer to a sample that comprises both wild type copies of a genomic locus and mutant copies of the genomic locus that have a point mutation relative to the wild type copies of the genomic locus, extending the tailed primer using the genomic locus as a template to produce a primer extension product and then detecting the primer extension product in a quantitative polymerase chain reaction (qPCR) assay that employs a forward primer that is complementary to a sequence in the complement of the 5′ tail of the tailed primer, a reverse primer, and a hydrolysis probe that is complementary to at least 6 nucleotides of the target complementary sequence of the tailed primer and at least 6 nucleotides of the 5′ tail of the tailed primer.

In some embodiments, the method may comprise combining the tailed primer, sample, forward primer, reverse primer, and hydrolysis probe with polymerase and nucleotides in a reaction vessel to produce a reaction mixture, and then thermocycling the reaction mixture without opening the reaction vessel or adding additional reagents to the reaction mixture. In these embodiments, the Tₘ of the target complementary sequence of the tailed primer may be lower than the Tₘs of the forward primer, the reverse primer and the hydrolysis probe. In these embodiments, the method may comprise subjecting the reaction mixture to the following thermocycling conditions: a first set of cycles that comprise a denaturation step followed by a first incubation at a temperature in the range of 40° C. to 52° C., a second incubation step at a temperature in the range of 55° C. to 65° C., a third incubation step at a temperature in the range of 65° C. to 75° C. and a second set of cycles that comprise a denaturation step followed by an incubation at one or more temperatures that are at least 8° C. higher than the temperature of the first incubation in the first set of cycles.

Kits for practicing the method are also provided.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates some of the principles of the present method.

FIG. 2 shows the mutation region within the in Squalene epoxidase gene of Trichophyton rubrum. Trichophyton rubrum: SEQ ID NO: 1, Trichophyton interdigitale: SEQ ID NO: 2, Trichophyton mentagrophytes: SEQ ID NO: 3, and Aspergillus niger: SEQ ID NO: 4.

FIGS. 3A-3C show a flow chart representing embodiments of the present disclosure.

FIGS. 4A-4D show the reagent stability for all the targets using Levey-Jennings plots. FIG. 4A: TRBDR RXN #1. FIG. 4B: TRBDR RXN #2. FIG. 4C: TRBDR RXN #3. FIG. 4D: TRBDR RXN #4.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Unless otherwise indicated terms and symbols of nucleic acid chemistry, biochemistry, genetics, and molecular biology used herein follow those of standard treatises and texts in the field, e.g. the Oxford Dictionary of Biochemistry and Molecular Biology (2 ed.) Ed. Richard Cammack (Oxford University Press, 2008) and The Dictionary of Cell and Molecular Biology 5th Edition Ed. L. M. Lackie (Academic Press, 2013).

The term “sample” as used herein relates to a material or mixture of materials, typically, although not necessarily, in liquid form, containing one or more analytes of interest.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “nucleic acid sample,” as used herein denotes a sample containing a nucleic acid or nucleic acids.

The term “target polynucleotide,” as used herein, refers to a polynucleotide of interest under study. In certain embodiments, a target polynucleotide contains one or more target sites that are of interest under study.

The term “oligonucleotide” as used herein denotes a single stranded multimer of nucleotides of from about 2 to 200 nucleotides. Oligonucleotides may be synthetic or may be made enzymatically, and, in some embodiments, are 10 to 100 nucleotides in length. Oligonucleotides may contain ribonucleotide monomers (i.e., may be oligoribonucleotides) or deoxyribonucleotide monomers. An oligonucleotide may be 10 to 20, 11 to 30, 31 to 40, 41 to 50, 51 to 60, 61 to 70, 71 to 80, 80 to 100, 100 to 150 or 150 to 200 nucleotides in length, for example.

The term “duplex,” or “duplexed,” as used herein, describes two complementary polynucleotides that are base-paired, i.e., hybridized together.

The term “primer” as used herein refers to an oligonucleotide that has a nucleotide sequence that is complementary to a region of a target polynucleotide. A primer binds to the complementary region and is extended, using the target nucleic acid as the template, under primer extension conditions. A primer may be in the range of about 15 to about 50 nucleotides although primers outside of this length may be used. A primer can be extended from its 3′ end by the action of a polymerase. An oligonucleotide that cannot be extended from it 3′ end by the action of a polymerase is not a primer.

The term “extending” as used herein refers to any addition of one or more nucleotides to the end of a nucleic acid, e.g. by ligation of an oligonucleotide or by using a polymerase.

The term “amplifying” as used herein refers to generating one or more copies of a target nucleic acid, using the target nucleic acid as a template.

The term “denaturing,” as used herein, refers to the separation of a nucleic acid duplex into two single strands.

The terms “determining”, “measuring”, “evaluating”, “assessing,” “assaying,” “detecting,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end.

As used herein, the term “Tₘ” refers to the melting temperature of an oligonucleotide duplex at which half of the duplexes remain hybridized and half of the duplexes dissociate into single strands. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

As used herein, the term “Tₘ-matched” refers to a plurality of nucleic acid duplexes having Tₘs that are within a defined range, e.g., within 5° C. or 10° C. of each other.

As used herein, the term “reaction mixture” refers to a mixture of reagents that are capable of reacting together to produce a product in appropriate external conditions over a period of time. A reaction mixture may contain PCR reagents and a hydrolysis probe, for example, the recipes for which are independently known in the art.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. A mixture is not addressable. To illustrate by example, an array of spatially separated surface-bound polynucleotides, as is commonly known in the art, is not a mixture of surface-bound polynucleotides because the species of surface-bound polynucleotides are spatially distinct and the array is addressable.

As used herein, the term “qPCR reagents” refers to all reagents that are required for performing a quantitative polymerase chain reaction (PCR) on a template. As is known in the art, PCR reagents essentially include a first primer, a second primer, a thermostable polymerase such as Taq polymerase or a variant thereof, and nucleotides and a hydrolysis probe. Depending on the polymerase used, ions (e.g., Mg²⁺) may also be present. PCR reagents may optionally contain a template from which a target sequence can be amplified.

As used herein, the term “hydrolysis probe” is a dual-labelled oligonucleotides, where the 5′ end of the oligonucleotide is labelled with a fluorescent reporter molecule while the 3′ end is labelled with a quencher molecule. The hydrolysis probe is designed so that the length of the sequence places the 5′ fluorophore and the 3′ quencher in close enough proximity so as to suppress fluorescence. In use, hydrolysis probes bind to a sequence between the binding sites for the PCR amplification primers. During the extension phase of the PCR cycle the DNA polymerase (typically Taq DNA polymerase, although others can be used (see, e.g., Kreuzer et al, Mol Cell Probes. 2000 14:57-60)) synthesizes the complementary strand downstream of the PCR primers. When extension reaches the bound hydrolysis probe the 5′-3′ exonuclease activity of the DNA polymerase degrades the hydrolysis probe. Cleavage of the probe separates the fluorescent reporter molecule from quencher, thereby allowing the reporter molecule to fluoresce. The DNA polymerase continues synthesizing the rest of the nascent strand, thus inclusion of the probe does not inhibit the PCR reaction. With subsequent PCR cycles the amount of fluorescent reporter released, and hence fluorescence, increases cumulatively.

As used herein, the term “genomic locus” refers to a defined region in a genome, i.e., a location. A genomic locus exists at the same location in the genomes of different cells of the same species or different individuals of the same species. A genomic locus in one cell or individual may have a nucleotide sequence that is identical or very similar (i.e., more than 99% identical) to the same genomic locus in a different cell or individual. The difference in nucleotide sequence between the same locus in different cells or individuals may be due to one or more nucleotide substitutions.

As used herein, a “site of a mutation” refers to the position of a nucleotide substitution in a genomic locus. Unless otherwise indicated, the site of a mutation in a nucleic acid can have a mutant allele or wild type allele of a mutation. The site of a mutation may be defined by genomic coordinates or coordinates relative to the start codon of a gene, for example.

As used herein, the term “point mutation” refers to the identity of the nucleotide present at a site of a mutation in the mutant copy of a genomic locus. The nucleotide may be on either strand of a double stranded DNA molecule.

As used herein, the term “wild type”, with reference a genomic locus, refers to the alleles of a locus that contain a wild type sequence. Other alleles of the locus may contain a mutation.

As used herein, the term “mutant”, with reference to a genomic locus, refers to the alleles of a locus that contain a mutant sequence. The mutant allele of a genomic locus may contain a nucleotide substitution that is not silent in that it that either alters the expression of a protein or changes the amino acid sequence of a protein, which causes a phenotypic change in the cells that are heterozygous or homozygous for the mutant sequence relative to cells containing the wild type sequence (depending on whether the mutation is dominant or recessive). Alternatively, the mutant allele of a genomic locus may contain a nucleotide substitution that is silent.

As used herein, the term “corresponds to” and grammatical equivalents thereof in the context of, for example, a nucleotide in an oligonucleotide that corresponds to a site of a mutation, is intended to identify the nucleotide that is correspondingly positioned relative to (i.e., positioned across from) a site of a mutation when two nucleic acids (e.g., an oligonucleotide and genomic DNA containing the mutation) are hybridized. Again, unless otherwise indicated (e.g., in the case of a nucleotide that “does not base pair” or “base pairs” with a point mutation) a nucleotide that corresponds to a site of a mutation may base pair with either the mutant or wild type allele of a sequence.

A sample that comprises “both wild type copies of a genomic locus and mutant copies of the genomic locus” and grammatical equivalents thereof, refers to a sample that contains multiple DNA molecules of the same genomic locus, where the sample contains both wild type copies of the genomic locus (which copies contain the wild type allele of the locus) and mutant copies of the same locus (which copies contain the mutant allele of the locus). In this context, the term “copies” is not intended to mean that the sequences were copied from one another. Rather, the term “copies” in intended to indicate that the sequences are of the same locus in different cells or individuals.

As used herein the term “nucleotide sequence” refers to a contiguous sequence of nucleotides in a nucleic acid. As would be readily apparent, the number of nucleotides in a nucleotide sequence may vary greatly. In particular embodiments, a nucleotide sequence (e.g., of an oligonucleotide) may be of a length that is sufficient for hybridization to a complementary nucleotide sequence in another nucleic acid. In these embodiments, a nucleotide sequence may be in the range of at least 10 to 50 nucleotides, e.g., 12 to 20 nucleotides in length, although lengths outside of these ranges may be employed in many circumstances.

As used herein the term “fully complementary to” in the context of a first nucleic acid that is fully complementary to a second nucleic acid refers to a case when every nucleotide of a contiguous sequence of nucleotides in a first nucleic acid base pairs with a complementary nucleotide in a second nucleic acid.

As used herein the term a “primer pair” is used to refer to two primers that can be employed in a polymerase chain reaction to amplify a genomic locus. A primer pair may in certain circumstances be referred to as containing “a first primer” and “a second primer” or “a forward primer” and “a reverse primer”. Use of any of these terms is arbitrary and is not intended to indicate whether a primer hybridizes to a top strand or bottom strand (or the coding strand or non-coding strand) of a double stranded nucleic acid.

Other definitions of terms may appear throughout the specification.

### DESCRIPTION OF EXEMPLARY EMBODIMENTS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In the following description, the skilled artisan will understand that any of a number of enzymes could be used in the methods, including without limitation, those isolated from thermostable or hyperthermostable prokaryotic, eukaryotic, or archaeal organisms. The skilled artisan will also understand that the enzymes that are used in the method include not only naturally occurring enzymes, but also recombinant enzymes that include enzymatically active fragments, cleavage products, mutants, and variants of wild type enzymes.

Method for Sample Analysis

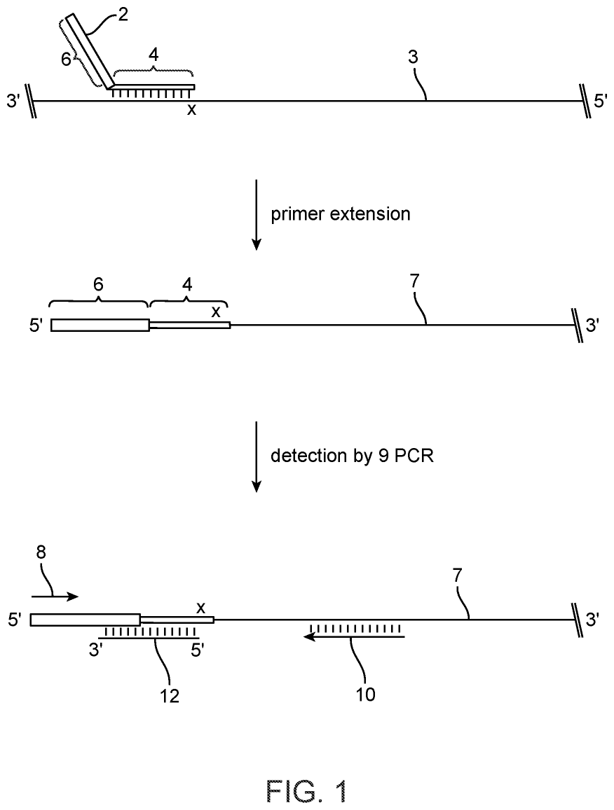

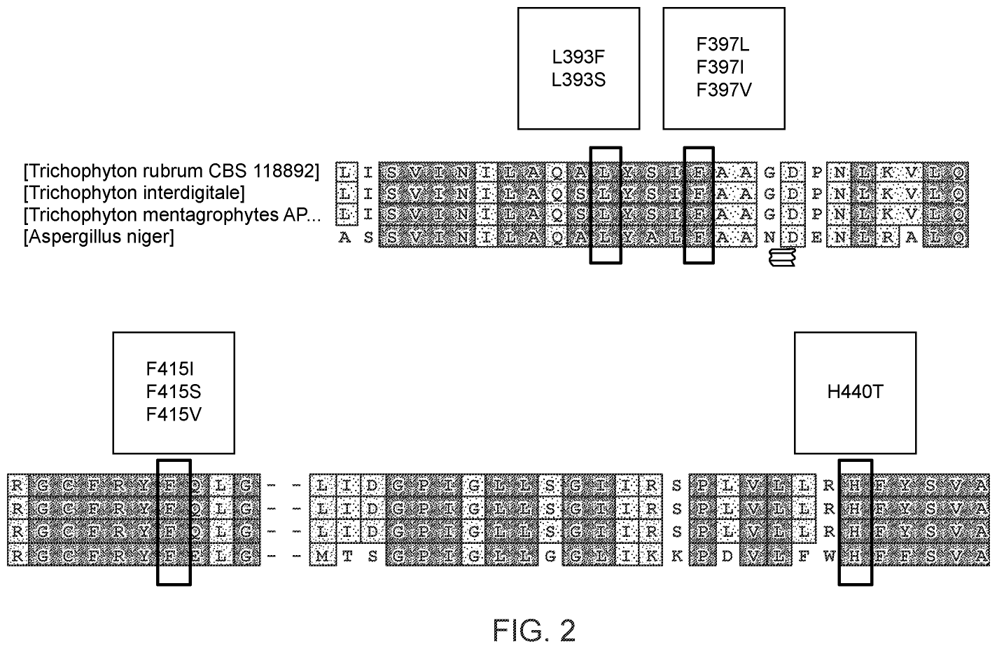

With reference to FIG. 1, some embodiments of the method may comprise hybridizing a tailed primer 2 to a sample that comprises both wild type copies of a genomic locus and mutant copies of the genomic locus that have a point mutation relative to the wild type copies of the genomic locus. FIG. 1 illustrates a mutant copy of the genomic locus 3, where the point mutation is indicated by an “x”. In some embodiments, the sample may contain at least 100 times (e.g., at least 1,000 times, at least 5,000 times, at least 10,000 times, at least 50,000 times or at least 100,000 times) more wild type copies of the genomic locus than mutant copies the genomic locus. As shown, the tailed primer 2 may comprise a 5′ tail 6 that is not complementary to the genomic locus as well as a target complementary sequence 4 that is fully complementary to a target site in the mutant locus including a 3′ terminal nucleotide that base pairs with the point mutation. That is, the tailed primer 2 comprises a 5′ tail and a target complementary sequence that has a 3′ end that base pairs with the point mutation. In some embodiments, target complementary sequence 4 of the tailed primer is no more than 26 nucleotides in length, e.g., in the range of 11-26 nucleotides. The 5′ tail 6 of tailed primer 2 may be at least 25 nucleotides (e.g., in the range of 30-50 nucleotides in length). After hybridizing the tailed primer to the sample, the method may comprise extending the tailed primer using the genomic locus as a template to produce a primer extension product 7.

Next, the method comprises detecting primer extension product 7 in a quantitative polymerase chain reaction (qPCR) assay. As shown in FIG. 1, the method assay may employ: (i) forward primer 8 that is complementary to a sequence in the complement of the 5′ tail of tailed primer (which is now part of primer extension product 7), (ii) a reverse primer 10 that hybridizes to a site in the genomic locus on the opposite strand and downstream from the site to which the tailed primer binds (and now hybridizes to primer extension product 7, as shown) and (iii) a hydrolysis probe 12. As shown, hydrolysis probe 12 is complementary to: i. at least 6 nucleotides of the target complementary sequence of the tailed primer and ii. at least 6 nucleotides of the 5′ tail of the tailed primer. In other words, the hydrolysis probe has a 5′ end that terminates at the 3′ of the tailed primer and is complementary to at least 6 nucleotides of the target complementary sequence of the tailed primer as well as several bases (e.g., at least 6 nucleotides) of the 5′ tail of the tailed primer. In some embodiments, the hydrolysis probe is complementary to 6, 8, 10, 12, 14 or 16 nucleotides of the target sequence of the tailed primer. In some embodiments, the hydrolysis probe is complementary to the entirety of the target complementary sequence of the tailed primer excluding 1-5 nucleotides from the 3′ end of the tailed primer. In some embodiments, the hydrolysis probe is complementary to the entirety of the target sequence of the tailed primer including the site of the point mutation, wherein, in some cases, the nucleotide at the 5′ end of the hydrolysis probe base pairs with the point mutation. The additional sequence added to the hydrolysis probe increases the Tm of the hydrolysis probe for the primer extension product relative to the genomic locus and allows the assay to be done in a single container by modulating the annealing temperatures of the reaction, without opening the container and without the addition of other reagents to the reaction during the reaction. In these embodiments, the target complementary sequence 4 of the tailed primer may be in the range of 11-26 nucleotides in length whereas the forward primer, reverse primer and hydrolysis probes may be at least 25 nucleotides in length. In some embodiments, the forward primer, reverse primer and hydrolysis probes may be Tₘ-matched.

In some embodiments, the method may comprises combining the tailed primer, sample, forward primer, reverse primer, and hydrolysis probe with polymerase and nucleotides in a reaction vessel to produce a reaction mixture and thermocycling the reaction mixture without opening the reaction vessel or adding additional reagents to the reaction mixture during the course of the reaction. In some embodiments, the Tₘ of the target complementary sequence of the tailed primer may be at least 8° C. lower (at least 9° C. lower, at least 10° C. lower, at least 11° C. lower or at least 12° C. lower) than each of the Tₘs of the forward primer, the reverse primer and the hydrolysis probe, thereby allowing the method to be implemented by subjecting the reaction mixture to the following thermocycling conditions: i. a first set of 1-5 cycles (e.g., 1-4 cycles, 1-3 cycles, or 1-2 cycles) that comprise a denaturation step followed by a first incubation at a temperature in the range of 40° C. to 52° C. which is then followed by a second incubation at temperature in the range of 55° C. to 65° C. followed by a third incubation step at a temperature in the range of 65° C. to 75° C. and ii. a second set of 20-50 cycles that comprise a denaturation step followed by an incubation at one or more temperatures that are at least 8° C. higher than the temperature of the first incubation in the first set of cycles. Fluorescence may be measured in each of the second set of cycles. For example, in some embodiments, the Tₘ of the target complementary sequence of the tailed primer may less than 52° C. and the Tₘs of each of the forward primer, the reverse primer and the hydrolysis probe may independently be at least 60° C. In this example, the method may comprise subjecting the reaction mixture to i. a first set of 1-5 cycles (e.g., 1-4 cycles, 1-3 cycles, or 1-2 cycles) of a first temperature of at least 90° C., a second temperature in the range of 40° C. to 52° C., a third temperature in the range of 55° C. to 65 and a fourth temperature in the range of 65° C. to 75° C.; followed by ii. a second set of 20-50 cycles of a fifth temperature of at least 90° C., a sixth temperature that is at least 8° C. higher than the second temperature, and an optional seventh temperature in the range of 65° C. to 75° C. Fluorescence may be measured in each of the second set of cycles. In other words, the second set of cycles can be implemented using “two step” or “three step” thermocycling conditions, which are known. Each of the cycles may be independently of a duration in the range of 10 seconds to 3 minutes, although durations outside of this range are readily employed. In each cycle of the second set of cycles (e.g., while the reaction is “extension” temperate), a signal generated by cleavage of the hydrolysis probe may be measured to provide a real-time measurement of the amount mutant nucleic acid in the sample. The increase in the amount of amplified product (indicated by the amount of fluorescence) can be measured in real-time, where the term “real-time” is intended to refer to a measurement that is taken as the reaction progresses and products accumulate. The measurement may be expressed as an absolute number of copies or a relative amount when normalized to a control nucleic acid in the sample.

As noted above, the hydrolysis probe should be designed to bind to a sequence in the primer extension product. The hydrolysis probe contains a fluorescent reporter dye attached to the 5′ end and a quencher dye that is attached to the 3′ end of the probe. The proximity of the two dyes inhibits the reporter from emitting fluorescence. The DNA polymerase used in the method should has 5′ to 3′ exonuclease (or flap endonuclease) activity which allows cleavage of the 5′ terminal nucleotide. Taq polymerase or any of its variants can be used in this method, although others are known. As the DNA polymerase amplifies the DNA strand extended from the reverse primer 10, it encounters the probe that is hybridized and cleaves the 5′ end of the hydrolysis probe. The DNA polymerase then cleaves the reporter from the probe, thereby releasing it from being quenched by the quencher (which is still on the hydrolysis probe). Fluorescence emitted by the reporter can be detected by the thermal cycler and recorded. Several different types of reporter dyes can be used in TaqMan probes, including FAM, TET, MAX, Atto550, CY5 or JOE, and different types of quenchers can be used as well, including TAMRA, BHQ and MGB. Choosing different reporters will depend on the instrumentation used for the experiment and whether the assay is being multiplexed, etc. Also, if different reporter dyes are used in multiplex experiments to detect multiple oligonucleotide sequences, the emission maxima (the peak of emission spectrum) of reporter dyes must have a difference of at least 15 nm. The amount of target in the sample can be quantified by standard curve analysis or by comparison to a control, for example.

The amount of product in the sample may be normalized relative to the amount of a control nucleic acid present in the sample, thereby determining a relative amount of the mutant copies in the sample. In some embodiments, the control nucleic acid may be a different locus to the genomic locus. In certain cases, the control nucleic acid may be detected using a qPCR assay that employs primers and probes that base pair with wild type copies of the genomic locus at the site of the point mutation, thereby detecting the presence of wild type copies of the genomic locus in the sample. The control may be measured in parallel with measuring the product in the same reaction mixture or a different reaction mixture. If the control is measured in the same reaction mixture, the assay may include further reagents, particularly a second tailed primer and a second hydrolysis probe that produces a signal that is distinguishable from the hydrolysis probe used to detect the mutant sequence. In particular embodiments, the reaction mixture may further other primers and probes for amplifying and detecting other mutations in the same locus or in a second genomic locus.

In certain cases, fluorescence indicating the amount of cleaved probe can be detected by an automated fluorometer designed to perform real-time PCR having the following features: a light source for exciting the fluorophore of the hydrolysis probe, a system for heating and cooling reaction mixtures and a fluorometer for measuring fluorescence by the released fluorophore. This combination of features, allows real-time measurement of the cleaved hydrolysis probe, thereby allowing the amount of target nucleic acid in the sample to be quantified. Automated fluorometers for performing real-time PCR reactions are known in the art and can be adapted for use in this specific assay, for example, the ICYCLER™ from Bio-Rad Laboratories (Hercules, Calif.), the Mx3000P™, the MX3005P™ and the MX4000™ from Stratagene (La Jolla, Calif.), the ABI PRISM™ 7300, 7500, 7700, and 7900 Taq Man (Applied Biosystems, Foster City, Calif.), the SMARTCYCLER™, ROTORGENE 2000™ (Corbett Research, Sydney, Australia) and the GENE XPERT™ System (Cepheid, Sunnyvale, Calif.) and the LIGHTCYCLER™ (Roche Diagnostics Corp., Indianapolis, Ind.). The speed of ramping between the different reaction temperatures is not critical and, in certain embodiments, the default ramping speeds that are preset on thermocyclers may be employed.

In certain cases, the method may further involve graphing the amount of cleavage that occurs in several cycles, thereby providing a real time estimate of the abundance of the nucleic acid target. The estimate may be calculated by determining the threshold cycle (i.e., the cycle at which this fluorescence increases above a predetermined threshold; the “Ct” value or “Cp” value). This estimate can be compared to a control (which control may be assayed in the same reaction mixture as the genomic locus of interest) to provide a normalized estimate. The thermocycler may also contain a software application for determining the threshold cycle for each of the samples. An exemplary method for determining the threshold cycle is set forth in, e.g., Luu-The et al (Biotechniques 2005 38: 287-293).

The sample used in the method may be from any source, including from a solid tissue or a bodily fluid such as blood, serum, plasma, saliva, mucous, phlegm, cerebral spinal fluid, pleural fluid, tears, lactal duct fluid, lymph, sputum, cerebrospinal fluid, synovial fluid, urine, amniotic fluid, and semen. In particular embodiments, a sample may be obtained from a subject, e.g., a human, and it may be processed prior to use in the subject assay. For example, the nucleic acid may be extracted from the sample prior to use, methods for which are known. For example, the sample may comprise cultured cells or a clinical sample, e.g., a tissue biopsy, scrape or lavage or cells of a forensic sample (i.e., cells of a sample collected at a crime scene). For example, in some embodiments, the sample may be made from a toenail, a fingernail, or portions thereof.

In some embodiments, the sample may be obtained from a human and the target complementary sequence of the tailed primer and the reverse primer may be complementary to a human genome. In these embodiments, the method may be to identify mutations (e.g., somatic mutations) in the genome. In other embodiments, the sample may be obtained from a human but the target complementary sequence of the tailed primer and the reverse primer may be complementary to a genome of a pathogen, e.g., a fungal, bacterial or viral pathogen. In these embodiments the point mutation may provide antibiotic resistance.

Reaction Mixture

The reaction mixture used in the method generally contains: (a) a sample that comprises both wild type copies of a genomic locus and mutant copies of the genomic locus that have a point mutation relative to the wild type copies of the genomic locus; (b) a tailed primer, wherein the tailed primer comprises: i. a target complementary sequence that is fully complementary to a target site in the genomic locus and includes a 3′ terminal nucleotide that base pairs with the point mutation in the locus; and ii. a 5′ tail that is not complementary to the genomic locus; (c) a forward primer that is complementary to a sequence in the complement of the 5′ tail of the tailed primer; (d) a reverse primer that hybridizes to a site in the genomic locus on the opposite strand and downstream from the site to which the tailed primer binds; and (e) a hydrolysis probe that is complementary to at least 6 nucleotides of the target complementary sequence of the tailed primer and at least 6 nucleotides of the 5′ tail of the tailed primer. Details of these components are described above. The reaction mixture is characterized in that it can amplify and detect the presence of mutant copies of a genomic locus in a background of wild type copies of the locus in the sample. Specifically, the reaction mixture used in the method may contain: a) amplification reagents comprising a thermostable polymerase (e.g., Taq polymerase or a variant thereof), nucleotides (e.g., dGTP, dATP, dTTP and dCTP), reaction buffer (which includes Mg²⁺), a tailed primer, a hydrolysis probe, first and second primers and a sample, as described above. Exemplary reaction buffers and DNA polymerases that may be employed in the subject reaction mixture include those described in various publications (e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). Reaction buffers and DNA polymerases suitable for PCR may be purchased from a variety of suppliers, e.g., Invitrogen (Carlsbad, CA), Qiagen (Valencia, CA) and Stratagene (La Jolla, CA). Guidance for the reaction components suitable for use with a polymerase as well as suitable conditions for their use is found in the literature supplied with the polymerase.

In particular embodiments, the reaction mixture may contain reagents for assaying multiple (e.g., at least 2, 3, 4 or more) different targets sequences in parallel. In these cases, the reaction mixture may contain multiple sets of primers/probe. The fluorophore may be selected from, e.g., 6-carboxyfluorescein (FAM), which has excitation and emission wavelengths of 485 nm and 520 nm respectively, MAX, which has excitation and emission wavelengths of 524 nm and 557 nm respectively, Atto550, which has excitation and emission wavelengths of 554 nm and 575 nm respectively, Redmond Red, which has excitation and emission wavelengths of 578 nm and 650 nm respectively and Yakima Yellow, which has excitation and emission wavelengths of 532 nm and 569 nm respectively, and Quasor670, which has excitation and emission wavelengths of 644 nm and 670 nm respectively, and CY5 which has excitation and emission wavelengths of 651 nm and 670 nm respectively, although many others could be employed. In certain cases, at least one of the primer/probe sets be for the detection of an internal control.

In a multiplex reaction, the various primers/probes may be designed to have similar thermodynamic properties, e.g., similar Tₘs, G/C content, and in certain embodiments some may all be of a similar length. The other reagents used in the reaction mixture may also be Tₘ matched.

The assay mixture may be present in a vessel, including without limitation, a tube; a multi-well plate, such as a 96-well, a 384-well, a 1536-well plate; and a microfluidic device. In certain embodiments, multiple multiplex reactions are performed in the same reaction vessel. Depending on how the reaction is performed, the reaction mixture may be of a volume of 5 μl to 200 μl, e.g., 10 μl to 100 μl, although volumes outside of this range are envisioned.

Kits

Also provided are kits for practicing the subject method, as described above. The components of the kit may be present in separate containers, or multiple components may be present in a single container. In particular embodiments, a kit may comprise: (a) a tailed primer, wherein the tailed primer comprises: i. a target complementary sequence that is fully complementary to a target site in a genomic locus and includes a 3′ terminal nucleotide that base pairs with a point mutation in the locus; and ii. a 5′ tail that is not complementary to the genomic locus; (b) a forward primer that is complementary to a sequence in the complement of the 5′ tail of the tailed primer; (c) a reverse primer that hybridizes to a site in the genomic locus on the opposite strand and downstream from the site to which the tailed primer binds; and (d) a hydrolysis probe that is complementary to at least 6 nucleotides of the target complementary sequence of the tailed primer and at least 6 nucleotides of the 5′ tail of the tailed primer. The particulars of these reagents are described above. The kit further comprises a primer/probe set for amplification and detection of a control nucleic acid. In addition to above-mentioned components, the kit may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate. In addition to the instructions, the kits may also include one or more control samples, e.g., positive or negative controls analytes for use in testing the kit.

Utility

The method described finds use in a variety of applications, where such applications generally include sample analysis applications in which the presence of a target nucleic acid sequence in a given sample is detected.

In particular, the above-described methods may be employed to diagnose a disease, to guide treatment, or to investigate a condition or disease. Many nucleotide mutations are associated with and are thought to be a factor in producing these disorders. Knowing the type and the location of the nucleotide polymorphism may greatly aid the diagnosis, prognosis, and understanding of various mammalian diseases. In addition, the assay conditions described herein can be employed in other nucleic acid detection applications including, for example, for the detection of infectious diseases, viral load monitoring, viral genotyping, environmental testing, food testing, forensics, epidemiology, and other areas where specific nucleic acid sequence detection is of use.

In one embodiment, a sample may be collected from a patient at a first location, e.g., in a clinical setting such as in a hospital or at a doctor's office, and the sample may be forwarded to a second location, e.g., a laboratory where it is processed and the above-described method is performed to generate a report. A “report” as described herein, is an electronic or tangible document which includes report elements that provide test results that may include a Ct value, or Cp value, or the like that indicates the presence of mutant copies of the genomic locus in the sample. Once generated, the report may be forwarded to another location (which may the same location as the first location), where it may be interpreted by a health professional (e.g., a clinician, a laboratory technician, or a physician such as an oncologist, surgeon, pathologist), as part of a clinical diagnosis.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### I. Utility

### A. Intended Utility

TABLE 1
List of Mutations (single nucleotide polymorphism (SNP)
and their Non-synonymous changes in the amino acid

| Gene | | | |
|---|---|---|---|
| position (bp) | Wild type | Target name | Point Mutations |
| 1177-1179 | TTA (Leu393) | TRB-1177M1 | TTT (Leu393Phe) |
| | | TRB-1177M2 | TCA (Leu393Ser) |
| | | TRB-1177M3 | TTC (Leu393Phe) |
| 1189-1191 | TTC (Phe397) | TRB-1189M1 | TTA (Phe397Leu) |
| | | TRB-1189M2 | CTC (Phe397Leu) |
| | | TRB-1189M3 | ATC (Phe397Ile) |
| | | TRB-1189M4 | GTC (Phe397Val) |
| | | TRB-1189M5 | TTG (Phe397Leu) |
| 1305-1307 | TTC (Phe415) | TRB-1305M1 | ATC (Phe415Ile) |
| | | TRB-1305M2 | GTC (Phe415Val) |
| | | TRB-1305M3 | TCC (Phe415Ser) |
| 1380-1382 | CAT (His440) | TRB-1380M | TAT (His440Tyr) |

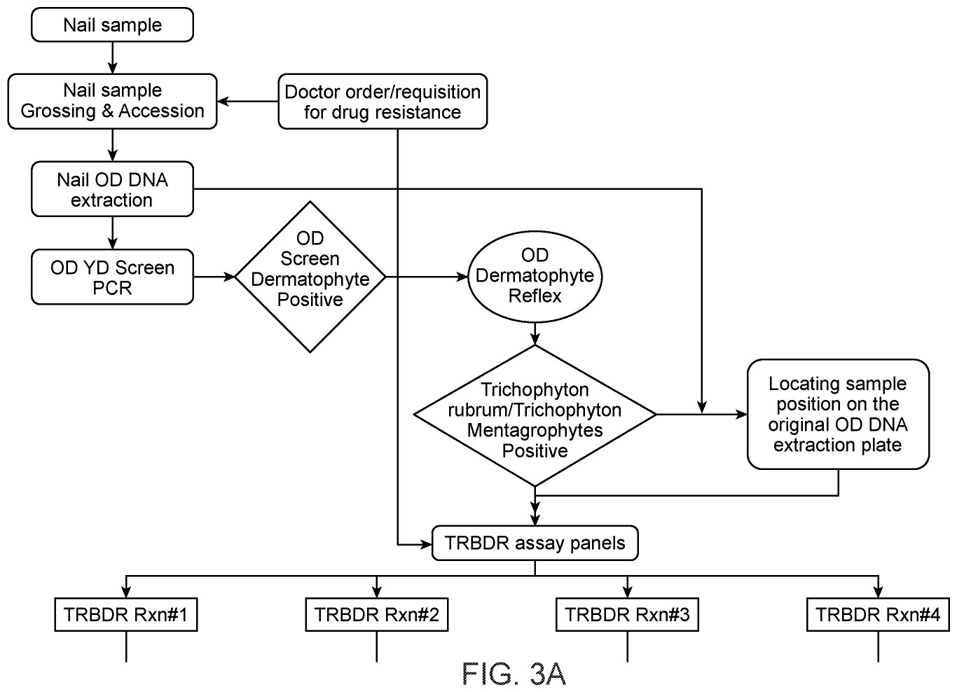

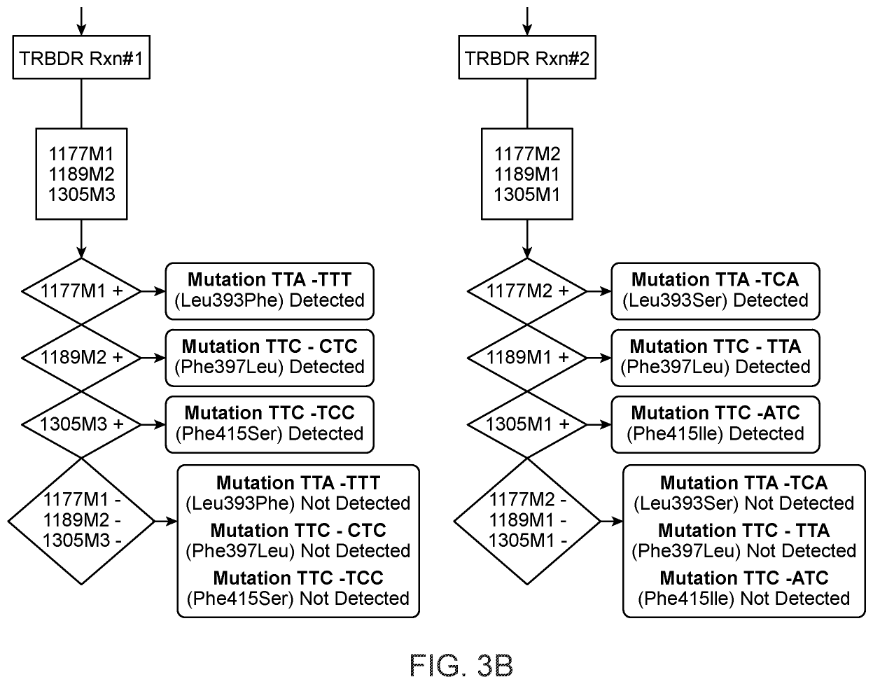

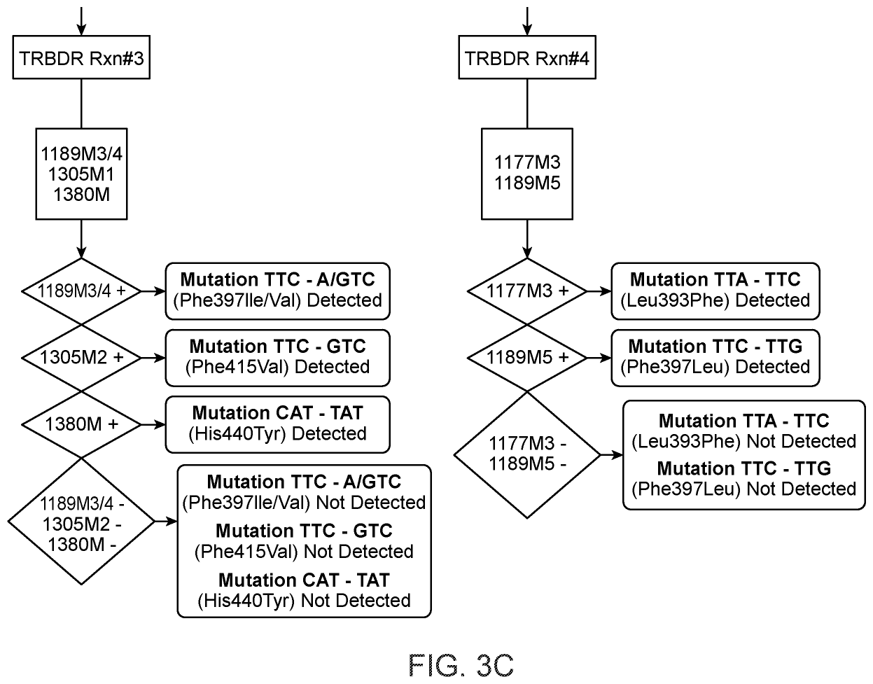

Specimens for the TRBDR assay are obtained from individuals suspected of onychomycosis and that were identified as T. rubrum or T. mentagrophytes using our Onychodystrophy Infectious Agent Detection (OIAD) screen and Dermatophyte Reflex assay. The workflow for the TRBDR assay is described in the flow chart in FIGS. 3A-3C.

The assay is intended for use only in accordance to the CAP regulations, and will be performed at, a CLIA certified high-complexity laboratory experienced in the development and performance of molecular diagnostic assays.

### B. Instruments Used with Test

1. DNA extraction:
a. Hamilton Microlab STAR systems (Hamilton Company, Reno, USA) are used for automated DNA extraction from clinical specimens.

2. Real-time PCR:
a. The TRBDR assay panel is performed with PCR using AmpliTaq Gold™ DNA Polymerase (ThermoFisher Scientific).
   i. QuantStudio 6 PCR systems (ThermoFisher Scientific), software version 1.3, with 384 well plate format are used for one-step RT-PCR analyses.
   ii. Hamilton Microlab STARlet systems (Hamilton Company, Reno, USA) are used for dispensing samples and master mixtures.

3. Reagents and Materials used with the test is shown in Table 1.

TABLE 2
List of key reagents used in the assay panel

| Reagent | Manufacturer | Sequence or Catalog # |
|---|---|---|
| Mag-Bind Plant DNA DS | Omega Bio-Tek, | M1130-01 |
| Kit | Inc, | |
| AmpliTaq Gold ™ DNA | Thermo Fisher | N8080241 |
| Polymerase | | |
| dNTP mix | Thermo Fisher | 4303441 |
| Positive Control plasmid | GeneScript | Customized synthesis |
| Trychophyton rubrum | In-house | DNA extraction from |
| ATCC 22402 DNA | | the ATCC culture |

TABLE 3
Primer/probe sequences and target regions employed in the TRBDR assay panel

| | | | |
|---|---|---|---|
| Mutation | Primer / Probe | Sequence | Fluorescence |

Table 3a: TRBDR reaction 1 (RXN#1)

| | | | |
|---|---|---|---|
| 1177-M1 | DTR-R1-9 | GGTGACAGATTAGTAATCCGCCAAGCGCGCATCA | |
| TTT(Leu393Phe) | | GGCTTGCAAGTCTCCGGCGAATATAGAGTAA | |
| | | (SEQ ID NO: 5) | |
| | DRT-R1-Probe4 | CTTGCAAGTCTCCGGCGAATATAGAG (SEQ ID | MAX |
| | | NO: 6) | |
| 1189-M2 | DTR-R1-8 | CGTGATTCATGCCGACCTTATATGACCGACAGACT | |
| CTC(Phe397Leu) | | GACCGAGCTTGCATTACCAGCGGCGAG (SEQ ID | |
| | | NO: 7) | |
| | DRT-R1-Probe3 | CGAGCTTGCATTACCAGCGGC (SEQ ID NO: 8) | FAM |
| 1305-M3 | DTR-R1-10 | GGTGACAGATTAGTAATCCGCCAAGCGCGCATCA | |
| TCC(Phe415Ser) | | GGTGTAAGCAGTGAGTCTATAAGTCCAAGTTGGG | |
| | | (SEQ ID NO: 9) | |
| | DRT-R1-Probe2 | AGGTGTAAGCAGTGAGTCTATAAGTCCAAGT | Atto550 |
| | | (SEQ ID NO: 10) | |
| common primers | DTR-R1-6 | GACAGATTAGTAATCCGCCAAGC (SEQ ID NO: 11) | |
| | DTR-R1-7 | GATTCATGCCGACCTTATATGACC (SEQ ID NO: 12) | |
| | DTR-R1-1 | TTCCACTGGCAACGGAAGTC (SEQ ID NO: 13) | |
| Control 1 | DTR-R1-2 | CGACGCCAACGATAAGATCCTG (SEQ ID NO: 14) | |
| | DTR-R1-3 | GGCACTGGACTCCAAGGAC (SEQ ID NO: 15) | |
| | DRT-R1-Probe1 | TCACCATTGTCGAGACCAAGGC (SEQ ID NO: 16) | CY5 |

Table 3b: TRBDR reaction 2 (RXN#2)

| | | | |
|---|---|---|---|
| 1177-M2 | DTR-R2-10 | GCTTTCCGAGTTGTGCAATCCAGATCTCGTTGGCT | |
| TCA(Leu393Ser) | | CCGCTTGCAAGTCTCCGGCGAATATAGAGTATG | |
| | | (SEQ ID NO: 17) | |
| | DRT-R2-Probe4 | CTTGCAAGTCTCCGGCGAATATAGAG (SEQ ID | MAX |
| | | NO: 18) | |
| 1189-M1 | DTR-R2-9 | ACGTACTGATTGGCAACCGGAGTGTATTGCAGAT | |
| TTA(Phe397Leu) | | GCAGAATATGTTGAGTCCACCAGCGGCT (SEQ ID | |
| | | NO: 19) | |
| | DRT-R2-Probe3 | ATGCAGAATATGTTGAGTCCACCAGC (SEQ ID | FAM |
| | | NO: 20) | |
| 1305-M1 | DTR-R2-11 | ACAGTGACGCAGAGGCTCTTGGTCTTTCAGCTCA | |
| ATC(Phe415Ile) | | GACGACAACGAGTAATCTATAAGTCCAAGTTGGA | |
| | | T (SEQ ID NO: 21) | |
| | DRT-R1-Probe2 | AGACGACAACGAGTAATCTATAAGTCCAAGTTG | Atto550 |
| | | (SEQ ID NO: 22) | |
| common primers | DTR-R2-6 | CCGAGTTGTGCAATCCAGATCT (SEQ ID NO: 23) | |
| | DTR-R2-7 | ACTGATTGGCAACCGGAGTG (SEQ ID NO: 24) | |
| | DTR-R2-8 | GACGCAGAGGCTCTTGGTC (SEQ ID NO: 25) | |
| | DTR-R1-1 | TTCCACTGGCAACGGAAGTC (SEQ ID NO: 26) | |
| Control 2 | DTR-R1-2 | CGACGCCAACGATAAGATCCTG (SEQ ID NO: 27) | |
| | DTR-R1-3 | GGCACTGGACTCCAAGGAC (SEQ ID NO: 28) | |
| | DRT-R1-Probe1 | TCACCATTGTCGAGACCAAGGC (SEQ ID NO: 29) | CY5 |

Table 3c: TRBDR reaction 3 (RXN#3)

| | | | |
|---|---|---|---|
| 1189-M3 | DTR-R3-8 | ACGTACTGATTGGCAACCGGAGTGTATTGCAGt | |
| ATC(Phe397Ile) & | | gtaagcagtgagtCCAGKCCTTATACTCTATAa (SEQ | |
| 1189-M4 | | ID NO: 30) | |
| GTC(Phe397Val) | | | |
| | DTR-R3-10 | ATCATGTAGTACGCGATTCAAGCTCTCTGAcaG | |
| | | agtctgtaagcagtgagtCCAGKCCTTATACTC | |
| | | TATAg (SEQ ID NO: 31) | |
| | DRT-R3-Probe2 | TGTAAGCAGTGAGTCCAGKCCTTATACT (SEQ | FAM |
| | | ID NO: 32) | |
| 1305-M2 | DTR-R3-11 | ACAGTGACGCAGAGGCTCTTGGTCTTTCAGCTC | |
| GTC(Phe415Val) | | AGACGACAACGAGTAATGCTGCTTCAGGTATG | |
| | | (SEQ ID NO: 33) | |
| | DRT-R3-Probe4 | AGACGACAACGAGTAATGCTGCTTCA (SEQ ID | Atto550 |
| | | NO: 34) | |
| 1380-M | DTR-R3-12 | GCTTTCCGAGTTGTGCAATCCAGATCTCGTTGG | |
| TAT(His440Tyr) | | CTCCGCTTGCAAGTCTCCCTTGGTTCTATTACG | |
| | | RT (SEQ ID NO: 35) | |
| | DRT-R3-Probe5 | CGCTTGCAAGTCTCCCTTGGTTCTATTA (SEQ ID | MAX |
| | | NO: 36) | |
| Common primers | DTR-R2-6 | CCGAGTTGTGCAATCCAGATCT (SEQ ID NO: | |
| | | 37) | |
| | DTR-R2-7 | ACTGATTGGCAACCGGAGTG (SEQ ID NO: 38) | |
| | DTR-R2-8 | GACGCAGAGGCTCTTGGTC (SEQ ID NO: 39) | |
| | DTR-R3-4 | ATGTAGTACGCGATTCAAGCTCT (SEQ ID NO: 40) | |
| | DTR-R3-5 | AGATGGGTTTGCTAGTAAGGTGTAG (SEQ ID NO: 41) | |
| Control 3 | DTR-R3-6 | CCTTGACCCTTTYCGAGTGCA(SEQ ID NO: 42) | |
| | DTR-R3-7 | TGTCTAGCTTTGAAGTTCGGCAA (SEQ ID NO: 43) | |
| | DRT-R3-Probe1 | CCTGCAGGGTCATCCTTCCTTTC (SEQ ID NO: 44) | CY5 |

Table 3d: TRBDR reaction 4 (RXN#4)

| | | | |
|---|---|---|---|
| 1177-M3 | DTR-R4-7 | GCTTTCCGAGTTGTGCAATCCAGATCTCGTTGG | |
| TTC(Leu393Phe) | | CTCTTGCAAGTCTCGCGGCGAATATAGAGTAG | |
| | | (SEQ ID NO: 45) | |
| | DRT-R2-Probe4 | CTTGCAAGTCTCGCGGCGAATATAGA (SEQ ID | MAX |
| | | NO: 46) | |
| 1189-M5 | DTR-R4-6 | ACAGTGACGCAGAGGCTCTTGGTCTTTCAGCTC | FAM |
| CTC(Phe397Leu) | | ACAGAACGAGTAATCCACCAGCGGCC (SEQ ID | |
| | | NO: 47) | |
| | DRT-R4-Probe3 | CACAGAACGAGTAATCCACCAGCG (SEQ ID | |
| | | NO: 48) | |
| Common primers | DTR-R1-6 | CCGAGTTGTGCAATCCAGATCT (SEQ ID NO: 49) | |
| | DTR-R2-8 | ACTGATTGGCAACCGGAGTG (SEQ ID NO: 50) | |
| | DTR-R1-1 | CGTTGATTTCTGTCATCAATATTCTTGC (SEQ ID | |
| | | NO: 51) | |
| Control 4 | DTR-R1-2 | CGACGCCAACGATAAGATCCTG (SEQ ID NO: 52) | CY5 |
| | DTR-R4-5 | GGCACTGGACTCCAAGGAC (SEQ ID NO: 53) | |
| | DRT-R1-ProbL3 | TCACCATTGTCGAGACCAAGGC (SEQ ID NO: 54) | |

### II. Example 1: Assay Description and Test Principle

### A. Product Overview/Test Principle

The TRBDR Assay panel is a molecular in vitro diagnostic test that aids in the detection of Terbinafine drug-resistant mutations in SQLE gene from nail specimens based on a proprietary methodology developed at which is based on the real-time PCR method using TaqMan Technology. The product contains oligonucleotide primers and dual-labeled hydrolysis probes (TaqMan®), and control material used in qPCR for the in vitro qualitative detection of mutant DNA in nail specimens.

TRBDR assay panel uses a mutation specific primer with extra Tag (randomly generated sequence with no known homology with other organisms) sequence and reverse primer for the pre-amplification step with annealing temperature at 49° C. for 5 cycles. In the second step for further amplification and to avoid non-specific amplification of the wild type sequence, primer specific to the Tag sequence of the initial mutant primer along with the reverse primer is used at annealing temperature at 60° C. for 40 cycles, and amplifies only the tagged pre-amplified mutant fragment. A specifically designed TaqMan probe covering both the Tag and the target sequence to further increase the specificity serves as signal for mutation detection.

The Terbinafine Drug Resistance (TRBDR) mutation detection assay panel includes four reactions which detects 12 mutations in Squalene epoxidase gene and each reaction include an endogenous control in the different region of the same gene. Each reaction contain primer/probe sets that target the 2-3 specific mutation regions.

TABLE 4
The configuration of TRBDR assay panel

| | Mutation | Mutation | Mutation | Endogenous |
|---|---|---|---|---|
| Panels | Target 1 | Target 2 | Target 3 | control Target |
| RXN#1 | TRB-1177M1 | TRB-1189M2 | TRB-1305M3 | TRB-Control1 |
| RXN#2 | TRB-1177M2 | TRB-1189M1 | TRB-1305M1 | TRB-Control2 |
| RXN#3 | TRB-1189M3/4 | TRB-1305M2 | TRB-1380M | TRB-Control3 |
| RXN#4 | TRB-1177M3 | TRB-1189M5 | | TRB-Control4 |

### B. Control Material(s)

1. PCR template controls: These controls are added to discrete wells on plates used for the real-time PCR step.
a. Positive template controls (PC): These controls ensure that the PCR are performing as intended and are used on every PCR plate. The PC is comprised of targets for each mutation. The PC includes Plasmid DNA for each of the reaction
   All the targets in a reaction must be detected within the specified Ct range to ensure proper performance of the assay.
b. Negative template control (NC): This is a no template control included to eliminate the possibility of cross-contamination during PCR steps, and is used for each of the reaction on every plate tested. The template used in this control is nuclease-free water of molecular grade.
   NC should be negative for all targets (Table 2).
   Negative reactions are required to assure proper performance of the assay.
c. Wild Type control (WC): This is a wild type sequence control included to exclude the possibility of detection of the non-specific amplification of wild type target during PCR steps, and is used for each of the reaction on every plate tested. The template used in this control is the wild type Trichophyton rubrum DNA extract.
WC should be either negative for mutant targets or serve as a cut-off for mutant target. (Table 2).
Wild Type control reactions are required to assure proper performance of the assay.

2. Endogenous Control:
a. TRBDR endogenous control: Each reaction has an endogenous control. This control is used to verify the quality and quantity of the Trichophyton DNA in the clinical specimen and its target sequence is a part of the SQLE gene indicating for the presence of Trichophyton in the sample. It serves as an amplification control for the mutant target detection in the same well.
   i. Endogenous control in each reaction needs to be amplified in clinical specimens that are called positive for the presence of specific mutation.
   ii. The absence of Endogenous control suggests no Trichophyton sps in the clinical sample.

3. Extraction negative control:
i. RB-Reagent blank: This control is processed through the extraction step and subsequent testing. RB consists of saline blank and monitors cross-contamination that may occur during the extraction process.
ii. RB should be negative for all targets.

C. Description of Test Steps:

1. Sample collection, transport, Sample accessioning and specimen processing Same as described in Onychodystrophy Infectious Agent Detection (OIAD) screen and Dermatophte Reflex assay, briefly:
a. Dry nail clippings were collected and transported at ambient temperature using a sealed bag or other sterile container with a tightly fitting cap.
b. Specimens were transported for receipt at within four days of collection.
c. The gross description for all specimens was recorded, to include the source, number of fragments, size and shape of submitted specimens.
d. Following gross analysis the nail samples are minced aseptically and portions of each are submitted and processed for histological analysis, PCR analysis and bacterial culture.
e. Minced nail samples were physically disrupted using ceramic bead homogenization.
f. EC/IC was added and samples were treated with detergent-based lysis buffer and digested with Proteinase K.
g. DNA was purified from the lysate and concentrated using the Mag-Bind® Plant DNA DS Kit (Omega Bio-tek) on an automated platform prior to PCR analysis.
h. Real-Time PCR was then performed with fluorescently labeled (TaqMan) probes to detect the presence of target organisms Amplification reactions were performed using Platinum II Taq Hot-Start DNA polymerase (Thermofisher) in PCR reaction amplified on the QuantStudio-6 PCR machine (Thermofisher). Screen primers were designed to specifically amplify DNA from 3 categories of fungal organisms: yeast, dermatophytes and saprophytes and one bacterium, Pseudomonas aeruginosa. Reflex primers were designed to specifically amplify organisms at the genus and/or species level.
   The OIAD Screen Assay consists of two separate PCR reactions:
   The OIAD Screen Rxn1 detects yeast and dermatophyte organisms and EC/IC.
   A dermatophyte target is determined to be positive in the sample according to Ct and delta Rn cutoff values established in the OIAD Screen Assay validation, then the OIAD dermatophyte Reflex Assay is performed.
i. The OIAD Reflex Assay consist of a separate PCR reaction:
   The OIAD Reflex Dermatophyte Rxn—detects Trichophyton rubrum complex, Trichophyton mentagrophytes complex, Epidermophyton spp., and Microsproum spp.
   A target is determined to be positive in the OIAD Reflex Dermatophyte for the sample according to Ct and delta Rn cutoff values established in this validation.
   The Trichophyton positive samples from OIAD Reflex Dermatophyte for the sample will be used for the TRBDR assay.

2. PCR procedure for TRBDR assay panel:
a. The total PCR reaction volume in each well is 10 ul.
b. Amplification and real-time PCR analysis is performed on the Applied Biosystems QuantStudio-6 Real-Time PCR System (Thermo Fisher Scientific, MA, US) with software configuration of 1.3.
c. PCR run conditions are:
   i. 95° C. for 10 min for enzyme activation
      Pre-amplification cycling (5 cycles):
   ii. 95° C. for 15 sec of denaturation
   iii. 49° C. for 30 sec of annealing
   iv. 60° C. for 30 seconds followed by 72° C. for 20 seconds for extension
      Amplification cycling (40 cycles):
   v. 95° C. for 15 sec of denaturation
   vi. 60° C. for 30 sec of annealing & Data acquisition
   vii. 72° C. for 20 seconds for extension
a. On each sample or RB control that has undergone extraction, 4 realtime-PCR reaction are performed for the detection of 12 mutations and respective endogenous controls of each reaction (see Table 4) in a 384-well PCR plate. In addition to the sample or RB controls, PCR template controls (PC, NC and WC) are added to separate wells on the 384-well PCR plate.
b. From each to be tested well of the 96-well DNA extracted plate, the Microlab STARlet workstation transfers 2 ul of an extracted sample or control to one well per reaction that contain 8 ul of master mixture component shown in Table 5 for the 4 reactions in a 384-well PCR plate.

TABLE 5
Components and their concentrations used for TaqMan PCR using
AmpliTaq Gold Taq polymerase chemistry in TRBDR assay panel.

| Component | Concentration/reaction | Volume/reaction | |
|---|---|---|---|
| TRBDR Specific Primers | 50X | 0.2 | ul |
| and Probes mix | | | |
| TRBDR enzyme Master | 3.5 X | 2.86 | ul |
| Mix | | | |
| PCR-grade water | | 4.94 | ul |
| 1X Master Mixture total | | 8 | ul |
| DNA (NC or PC or RB | | 2 | ul |
| control or clinical sample) | | | |
| PCR reaction Total | | 10 | ul |
| Volume | | | |

3. Data analysis and export for validated PCR Engine (interpretive software)
a. At completion of each PCR run, the QuantStudio-6 analyzes the run/data and calculates Ct values for individual targets using the baseline threshold settings as shown in Table 6.

TABLE 6
Baseline threshold setting for the assay
mutation targets and endogenous controls.

| Reaction | Target Name | Reporter | Threshold |
|---|---|---|---|
| TRBDR | TRB-1177M1 | MAX | 0.1 |
| Rxn#1 | TRB-1189M2 | FAM | 0.5 |
| | TRB-1305M3 | ATTO550 | 0.5 |
| | TRB-Ctrl1 | CY5 | 0.1 |
| TRBDR | TRB-1177M2 | MAX | 0.25 |
| Rxn#2 | TRB-1189M1 | FAM | 0.3 |
| | TRB-1305M1 | ATTO550 | 0.2 |
| | TRB-Ctrl2 | CY5 | 0.05 |
| TRBDR | TRB-1189M3/4 | FAM | 0.2 |
| Rxn#3 | TRB-1305M2 | ATTO550 | 0.15 |
| | TRB-1380M | MAX | 0.3 |
| | TRB-Ctrl3 | CY5 | 0.1 |
| TRBDR | TRB-1177M3 | MAX | 0.15 |
| Rxn#4 | TRB-1189M5 | FAM | 0.5 |
| | TRB-Ctrl4 | CY5 | 0.1 |

a. At the completion of an analytical run, technologists review the amplification plots for all TaqMan PCR reactions, and thereafter save and exports the data to an excel file.
b. The excel file is uploaded to PCR Engine. The engine first examines Ct values for controls (PC/NC/WC/RB) against cut-off values (QC parameters) stored in the PCR Engine. The acceptable Ct range for each control is shown in Table 7.

TABLE 7
PC, NC, and RB acceptance Ct ranges criteria
for each mutations and endogenous controls.

| | | Positive control | | |
|---|---|---|---|---|
| | NTC Ct | (POS_Ctrl) | | POS_Ctrl Ct |
| Reaction | cut-off | name | Target Name | Range |
| TRBDR | >38 | TRBDR | TRB-1177M1 | 24-28 |
| Rxn#1 | >38 | POS1a or b | TRB-1189M2 | 27-32 |
| | >38 | | TRB-1305M3 | 28-34 |
| | >38 | | TRB-Ctrl1 | 22-27 |
| TRBDR | >38 | TRBDR | TRB-1177M2 | 25-30 |
| Rxn#2 | >31.7 | POS2a or b | TRB-1189M1 | 24-29 |
| | >38 | | TRB-1305M1 | 26-32 |
| | >38 | | TRB-Ctrl2 | 21-26 |
| TRBDR | >38 | TRBDR | TRB- | 27-33 |
| Rxn#3 | | POS3a or b | 1189M3/4 | |
| | >38 | | TRB-1305M2 | 26-31 |
| | >34.5 | | TRB-1380M | 30-36 |
| | >38 | | TRB-Ctrl3 | 23-28 |
| TRBDR | >36 | TRBDR | TRB-1177M3 | 25.5-30.5 |
| Rxn#4 | >38 | POS4a or b | TRB-1189M5 | 26-31 |
| | >38 | | TRB-Ctrl4 | 24-28 |

c. If the controls Ct values are acceptable, the PCR Engine calculates delta Ct for each mutation target by subtracting the corresponding endogenous control Ct from a specific mutation target Ct for a sample (Table 8). Then the PCR Engine analyzes the data for each clinical specimen using both the Ct cut-off and the delta Ct cut-off for each mutation as indicated in the Table 9.

TABLE 8
Calculation of delta Ct (dCt) for each mutation target of a sample using the
mutant Ct and the endogenous control Ct values in a particular reaction.

| Reaction | Target Name | Ct | delta Ct (dCt) |
|---|---|---|---|
| TRBDR | TRB-1177M1 | TRB-1177M1 Ct | TRB-1177M1 Ct - TRB-Ctrl1 Ct |
| Rxn#1 | TRB-1189M2 | TRB-1189M2 Ct | TRB-1189M2 Ct - TRB-Ctrl1 Ct |
| | TRB-1305M3 | TRB-1305M3 Ct | TRB-1305M3 Ct - TRB-Ctrl1 Ct |
| | TRB-Ctrl1 | TRB-Ctrl1 Ct | |
| TRBDR | TRB-1177M2 | TRB-1177M2 Ct | TRB-1177M2 Ct - TRB-Ctrl2 Ct |
| Rxn#2 | TRB-1189M1 | TRB-1189M1 Ct | TRB-1189M1 Ct - TRB-Ctrl2 Ct |
| | TRB-1305M1 | TRB-1305M1 Ct | TRB-1305M1 Ct - TRB-Ctrl2 Ct |
| | TRB-Ctrl2 | TRB-Ctrl2 Ct | |
| TRBDR | TRB-1189M3/4 | TRB-1189M3/4 Ct | TRB-1189M3/4 Ct - TRB-Ctrl3 Ct |
| Rxn#3 | TRB-1305M2 | TRB-1305M2 Ct | TRB-1305M2 Ct - TRB-Ctrl3 Ct |
| | TRB-1380M | TRB-1380M Ct | TRB-1380M Ct - TRB-Ctrl3 Ct |
| | TRB-Ctrl3 | TRB-Ctrl3 Ct | |
| TRBDR | TRB-1177M3 | TRB-1177M3 Ct | TRB-1177M3 Ct - TRB-Ctrl4 Ct |
| Rxn#4 | TRB-1189M5 | TRB-1189M5 Ct | TRB-1189M5 Ct - TRB-Ctrl4 Ct |
| | TRB-Ctrl4 | TRB-Ctrl4 Ct | |

TABLE 9
Ct and delta Ct cut off criteria for each mutations and endogenous
control targets from a sample (TRBDR Assay panel).

| | | Mutant CT | delta CT |
|---|---|---|---|
| Rxn | Target | Cut-off | (dCt) cut-off |
| TRBDR Rxn#1 | TRB-1177M1 | 33 | 7.5 |
| | TRB-1189M2 | 34.5 | 15 |
| | TRB-1305M3 | 38 | 18 |
| | TRB-Ctrl1 | | |
| TRBDR Rxn#2 | TRB-1177M2 | 33.6 | 11 |
| | TRB-1189M1 | 31.7 | 8.5 |
| | TRB-1305M1 | 38 | 17.5 |
| | TRB-Ctrl2 | | |
| TRBDR Rxn#3 | TRB- | 38 | 16.5 |
| | 1189M3/M4 | | |
| | TRB-1305M2 | 33 | 15 |
| | TRB-1380M | 34.5 | 10 |
| | TRB-Ctrl3 | | |
| TRBDR Rxn#4 | TRB-1177M3 | 36 | 10 |
| | TRB-1189M5 | 35 | 9.2 |
| | TRB-Ctrl4 | | |

d. Examination and interpretation of results are indicated as below:
   i. A TERBINAFINE RESISTANT MUTATION Detected: Samples that show amplification for TRBDR assay mutation and endogenous target and below the Ct and dCt cut-off threshold.
   ii. TERBINAFINE RESISTANT MUTATION NOT Detected: Samples that show no amplification for TRBDR assay mutation and endogenous target and below the Ct and dCt cut-off threshold.
   iii. INVALID: Samples that fail to show result for TRBDR assay mutation and endogenous target.
   iv. Interpretation of the results, reporting, and actions are as shown in the Table 10.

TABLE 10
Matrix used for interpretation of the results, reporting, and further actions

| Wild type | | | | Result and Interpretation | |
|---|---|---|---|---|---|
| Codon | TRBDR Assay | | Amino Acid | Gene Mutation | Gene Mutation |
| (amino acid) | Target Name | Gene Mutation | Substitution | Detected | Not Detected |
| TTA | TRB-1177M1 | TTA→TTT | Leu393Phe | Terbinafine drug | Terbinafine drug |
| (Leu393) | | | | resistance gene | resistance gene |
| | | | | mutation TTA→TTT in | mutation |
| | | | | Squalene epoxidase | TTA→TTT (for the |
| | | | | gene is DETECTED | amino acid codon |
| | | | | which results in the | Leu393) in |
| | | | | amino acid Leu393Phe | Squalene epoxidase |
| | | | | substitution in Squalene | gene is NOT |
| | | | | epoxidase | detected |
| | TRB-1177M2 | TTA→TCA | Leu393Ser | Terbinafine drug | Terbinafine drug |
| | | | | resistance gene | resistance gene |
| | | | | mutation TTA→TCA | mutation |
| | | | | in Squalene epoxidase | TTA→TCA (for |
| | | | | gene is DETECTED | the amino acid |
| | | | | which results in the | codon Leu393) in |
| | | | | amino acid Leu393Ser | Squalene epoxidase |
| | | | | substitution in Squalene | gene is NOT |
| | | | | epoxidase | detected |
| | TRB-1177M3 | TTA→TTC | Leu393Phe | Terbinafine drug | Terbinafine drug |
| | | | | resistance gene | resistance gene |
| | | | | mutation TTA→TTC | mutation |
| | | | | in Squalene epoxidase | TTA→TTC (for the |
| | | | | gene is DETECTED | amino acid codon |
| | | | | which results in the | Leu393) in |
| | | | | amino acid Leu393Phe | Squalene epoxidase |
| | | | | substitution in Squalene | gene is NOT |
| | | | | epoxidase | detected |
| TTC | TRB-1189M1 | TTC→TTA | Phe397Leu | Terbinafine drug | Terbinafine drug |
| (Phe397) | | | | resistance gene | resistance gene |
| | | | | mutation TTC→TTA | mutation |
| | | | | in Squalene epoxidase | TTC→TTA (for the |
| | | | | gene is DETECTED | amino acid codon |
| | | | | which results in the | Phe397) in |
| | | | | amino acid Phe397Leu | Squalene epoxidase |
| | | | | substitution in Squalene | gene is NOT |
| | | | | epoxidase | detected |
| | TRB-1189M2 | TTC→CTC | Phe397Leu | Terbinafine drug | Terbinafine drug |
| | | | | resistance gene | resistance gene |
| | | | | mutation TTC→CTC | mutation |
| | | | | in Squalene epoxidase | TTC→CTC (for the |
| | | | | gene is DETECTED | amino acid codon |
| | | | | which results in the | Phe397) in |
| | | | | amino acid Phe397Leu | Squalene epoxidase |
| | | | | substitution in Squalene | gene is NOT |
| | | | | epoxidase | detected |
| | TRB-1189M3/4 | TTC→A/GTC | Leu393Ile/Val | Terbinafine drug | Terbinafine drug |
| | | | | resistance gene | resistance gene |
| | | | | mutation | mutation |
| | | | | TTC→A/GTC in | TTC→A/GTC (for |
| | | | | Squalene epoxidase | the amino acid |
| | | | | gene is DETECTED | codon Leu393) in |
| | | | | which results in the | Squalene epoxidase |
| | | | | amino acid Leu393Ile | gene is NOT |
| | | | | substitution in Squalene | detected |
| | | | | epoxidase | |
| | TRB-1189M5 | TTC→TTG | Phe397Leu | Terbinafine drug | Terbinafine drug |
| | | | | resistance gene | resistance gene |
| | | | | mutation TTC→TTG | mutation |
| | | | | in Squalene epoxidase | TTC→TTG (for the |
| | | | | gene is DETECTED | amino acid codon |
| | | | | which results in the | Phe397) in |
| | | | | amino acid Phe397Leu | Squalene epoxidase |
| | | | | substitution in Squalene | gene is NOT |
| | | | | epoxidase | detected |
| TTC | TRB-1305M1 | TTC→ATC | Phe415Ile | Terbinafine drug | Terbinafine drug |
| (Phe415) | | | | resistance gene | resistance gene |
| | | | | mutation TTC→ATC | mutation |
| | | | | in Squalene epoxidase | TTC→ATC (for |
| | | | | gene is DETECTED | the amino acid |
| | | | | which results in the | codon Phe415) in |
| | | | | amino acid Phe415Ile | Squalene epoxidase |
| | | | | substitution in Squalene | gene is NOT |
| | | | | epoxidase | detected |
| | TRB-1305M2 | TTC→GTC | Phe415Val | Terbinafine drug | Terbinafine drug |
| | | | | resistance gene | resistance gene |
| | | | | mutation TTC→GTC | mutation |
| | | | | in Squalene epoxidase | TTC→GTC (for |
| | | | | gene is DETECTED | the amino acid |
| | | | | which results in the | codon Phe415) in |
| | | | | amino acid Phe415Val | Squalene epoxidase |
| | | | | substitution in Squalene | gene is NOT |
| | | | | epoxidase | detected |
| | TRB-1305M3 | TTC→TCC | Phe415Ser | Terbinafine drug | Terbinafine drug |
| | | | | resistance gene | resistance gene |
| | | | | mutation TTC→TCC | mutation |
| | | | | in Squalene epoxidase | TTC→TCC (for the |
| | | | | gene is DETECTED | amino acid codon |
| | | | | which results in the | Phe415) in |
| | | | | amino acid Phe415Ser | Squalene epoxidase |
| | | | | substitution in Squalene | gene is NOT |
| | | | | epoxidase | detected |
| CAT | TRB-1380M | CAT→TAT | His440Tyr | Terbinafine drug | Terbinafine drug |
| (His440) | | | | resistance gene | resistance gene |
| | | | | mutation CAT-TAT | mutation |
| | | | | in Squalene epoxidase | CAT→TAT (for |
| | | | | gene is DETECTED | the amino acid |
| | | | | which results in the | codon His440) in |
| | | | | amino acid His440Tyr | Squalene epoxidase |
| | | | | substitution in Squalene | gene is NOT |
| | | | | epoxidase | detected |

### III. Example 2: Performance Evaluation

Dermatophytic Isolates for the terbinafine drug resistance is obtained from Belgium coordinated collection of micro-organisms (BCCM) (http://brionet.eu/catalogues/ihem-species-details?NUM=609&FIRSTITEM=1&RANGE=100), based on the published manuscript (5). The isolates are re-tested for terbinafine drug resistance by plating the isolates on the sabouraud dextrose agar (SDA)/Potato dextrose agar (PDA) plates containing different concentration of terbinafine. Reagent-grade terbinafine (TRB) were obtained in the powder form (Sigma-Aldrich, Missouri, USA). Drug stock solutions were prepared in dimethyl sulfoxide (DMSO). The dermatophyte isolates were cultured on SDA/PDA for 21-30 days. In vitro testing of the susceptibility to Terbinafine allylamine was performed according to the Clinical and Laboratory Standards Institute (CLSI) document M38-A3 (CLSI, 2018). The drugs were analyzed at the final concentration in the range of 0.002-32 μg/ml for TRB. Drug resistance mutation in the squalene epoxidase (SQLE) gene is confirmed by extracting the DNA from the isolates, amplifying the Squalene epoxidase gene and Sanger sequencing. Either the partial fragment harboring all the published mutations or the full length SQLE gene, was amplified and sequenced with the SQLE-F1 partial (5′-TTCCACTGGCAACGGAAGTC-3′; SEQ ID NO: 77), SQLE-R1-partial (5′-AGATGGGTTTGCTAGTAAGGTGTAG-3′; SEQ ID NO: 78), SQLE-F1 full (5′-ATGGTTGTAGAGGCTCCTCCC-3′; SEQ ID NO: 79), and SQLE-R1-full (5′-CTAGCTTTGAAGTTCGGCAAATAYGAA-3′; SEQ ID NO 80:) primer pairs respectively.11 ABI7500 PCR machine was run according to a program: 2 min 94° C.; 35 cycles×[15 s 94° C., 30 s 58° C., 1 min 72° C.]; 5 min 72° C.5 The obtained sequences of SQLE gene in all tested isolates were compared with the reference sequence for T. rubrum and T. mentagrophytes.

Two clinical isolates Bako040B and Bako337A are from Bako, obtained and isolated by culturing the nail specimens on SDA plates containing terbinafine. Isolates confirmed to have drug resistance mutation by Sanger sequencing. Table 11 shows the isolates used in TRBDR Assay panel for verification and validation.

Wild type strains for T rubrum (ATCC-22402) and T mentagrophytes (ATCC-28146) are obtained from ATCC. These isolates were confirmed to be terbinafine susceptible strains by culture and no mutations in squalene epoxidase gene by Sanger sequencing.

TABLE 11
Terbinafine drug resistance Isolates used in this study

| | | | | | | |
|---|---|---|---|---|---|---|
| | | isolation: | | | | |
| | scientific | host | | | | |
| | name | animal/ | | SQLE | Mutation | |
| Isolate ID | organism | body part | pathology | mutation | site | SNP |

from BCCM

| | | | | | | |
|---|---|---|---|---|---|---|
| IHEM27855 | T. rubrum | human | Tinea pedis | L393F | TRB-1177M1 | TTT |
| | | foot | | | | (Leu393Phe) |
| IHEM27866 | T. rubrum | human nail | Tinea | L393F | TRB-1177M1 | TTT |
| | | | unguium | | | (Leu393Phe) |
| IHEM27856 | T. rubrum | human | Tinea pedis | L393F | TRB-1177M1 | TTT |
| | | foot | | | | (Leu393Phe) |
| IHEM27864 | T. rubrum | human | Tinea pedis | L393F | TRB-1177M1 | TTT |
| | | foot | | | | (Leu393Phe) |
| IHEM27878 | T. rubrum | human nail | Tinea | L393F | TRB-1177M1 | TTT |
| | | | unguium | | | (Leu393Phe) |
| IHEM27859 | T. rubrum | human nail | Tinea | L393S | TRB-1177M2 | TCA |
| | | | unguium | | | (Leu393Ser) |
| IHEM27877 | T. rubrum | human nail | Tinea | L393S | TRB-1177M2 | TCA |
| | | | unguium | | | (Leu393Ser) |
| IHEM27871 | T. rubrum | human nail | Tinea | L393F | TRB-1177M3 | TTC |
| | | | unguium | | | (Leu393Phe) |
| IHEM27872 | T. rubrum | human nail | Tinea | L393F | TRB-1177M3 | TTC |
| | | | unguium | | | (Leu393Phe) |
| IHEM27867 | T. interdigitale | human nail | Tinea | F397L | TRB-1189M2 | CTC |
| | | | unguium | | | (Phe397Leu) |
| IHEM27869 | T. interdigitale | human nail | Tinea | F397L | TRB-1189M2 | CTC |
| | | | unguium | | | (Phe397Leu) |
| IHEM27858 | T. rubrum | human nail | Tinea | F397L | TRB-1189M2 | CTC |
| | | | unguium | | | (Phe397Leu) |
| IHEM27863 | T. rubrum | human | Tinea pedis | F397L | TRB-1189M2 | CTC |
| | | foot | | | | (Phe397Leu) |
| IHEM27870 | T. rubrum | human nail | Tinea | F397L | TRB-1189M2 | CTC |
| | | | unguium | | | (Phe397Leu) |
| IHEM27875 | T. rubrum | human nail | Tinea | F397I | TRB-1189M3 | ATC |
| | | | unguium | | | (Phe397Ile) |
| IHEM27862 | T. rubrum | human leg | Tinea | F397I | TRB-1189M3 | ATC |
| | | | corporis | | | (Phe397Ile) |
| IHEM27861 | T. rubrum | human nail | Tinea | F397V | TRB-1189M4 | GTC |
| | | | unguium | | | (Phe397Val) |
| IHEM27874 | T. interdigitale | human nail | Tinea | F397L | TRB-1189M5 | TTG |
| | | | unguium | | | (Phe397Leu) |
| IHEM27873 | T. rubrum | human nail | Tinea | F397L | TRB-1189M5 | TTG |
| | | | unguium | | | (Phe397Leu) |
| IHEM27860 | T. rubrum | human nail | Tinea | F415I | TRB-1305M1 | ATC |
| | | | unguium | | | (Phe415Ile) |
| IHEM27854 | T. rubrum | human nail | Tinea | F415V | TRB-1305M2 | GTC |
| | | | unguium | | | (Phe415Val) |
| IHEM27865 | T. rubrum | human | Tinea pedis | F415S | TRB-1305M3 | TCC |
| | | foot | | | | (Phe415Ser) |
| IHEM27868 | T. rubrum | human | Tinea pedis | H440Y | TRB-1380M | TAT |
| | | foot | | | | (His440Tyr) |
| IHEM27879 | T. rubrum | human nail | Tinea | H440Y | TRB-1380M | TAT |
| | | | unguium | | | (His440Tyr) |

from Bako Diagnostics

| | | | | | | |
|---|---|---|---|---|---|---|
| Bako-040B | T. rubrum | human nail | Tinea | L393S | TRB-1177M2 | TCA |
| | | | unguium | | | (Leu393Ser) |
| Bako-337A | T. rubrum | human nail | Tinea | F397L | TRB-1189M1 | TTA |
| | | | unguium | | | (Phe397Leu) |

### A. Limit of Detection (LoD)-Analytical Sensitivity:

The limit of detection (LoD) is defined as the lowest detectable concentration of T rubrum and T mentagrophytes DNA with a probability of 95% or greater detected at the lowest detectable concentration.

To mimic the clinical physiological scenarios of mixed infections of both mutant and wild type strains in the patient sample. In the LoD studies, different percentages ranging from 100% to 0.1% of T rubrum or T mentagrophytes mutant DNA was mixed with wild type quantified DNA at different total DNA concentrations and spiked into negative nail matrix. DNA was extracted using the method described in the Onychodystrophy Infectious Agent Detection (OIAD) screen and Dermatophte Reflex assay. Extracted DNA is tested for all the TRBDR assay for the mutation detection in four reactions. The preliminary LoD was determined for each mutation as the percentages of the DNA mixtures at a given concentration of total DNA. Further, 13 replicates of the samples spiked with three percentages around the preliminary LoD at three total DNA concentration was carried out to determine the LoD concentration for each mutation. Table 12 shows LoD for TRBDR Assay panel for each mutation.

TABLE 12
Analytical sensitivity studies for TRBDR Assay panel for each mutation

| | | | Total DNA | | | | | |
|---|---|---|---|---|---|---|---|---|
| | | | level | Mutant | | | | |
| Rxn | Mutation | Isolate# | (ng/Rxn) | DNA % | #Detected/Total | Ct Mean | dCt | dRn |
| Rxn1 | 1177M1 | IHEM27864 | 0.005 | 25% | 13/13 | 32.1 | 4.8 | 0.9 |
| | | | 0.05 | 5% | 13/13 | 31.9 | 7.4 | 0.9 |
| | | | 0.5 | 1% | 13/13 | 30.7 | 10.0 | 0.9 |
| | 1189M2 | IHEM27863 | 0.005 | 50% | 13/13 | 32.8 | 5.4 | 1.6 |
| | | | 0.05 | 10% | 13/13 | 31.4 | 7.1 | 1.9 |
| | | | 0.5 | 1% | 13/13 | 31.0 | 10.4 | 2.1 |
| | 1305M3 | IHEM27865 | 0.005 | 50% | 13/13 | 32.8 | 4.8 | 1.5 |
| | | | 0.05 | 5% | 13/13 | 33.2 | 8.8 | 1.4 |
| | | | 0.5 | 1% | 13/13 | 32.2 | 11.6 | 1.3 |
| Rxn2 | 1177M2 | Bako-040B | 0.005 | 50% | 13/13 | 36.1 | 10.4 | 1.4 |
| | | | 0.05 | 5% | 13/13 | 36.3 | 13.5 | 1.1 |
| | | | 0.5 | 0.2% | 13/13 | 34.0 | 14.5 | 0.5 |
| | 1189M1 | Bako-337A | 0.005 | 50% | 13/13 | 36.0 | 9.7 | 0.3 |
| | | | 0.05 | 5% | 13/13 | 37.7 | 14.8 | 0.3 |
| | | | 0.5 | 1% | 13/13 | 34.4 | 15.1 | 0.4 |
| | 1305M1 | IHEM27860 | 0.005 | 25% | 13/13 | 35.7 | 10.4 | 0.3 |
| | | | 0.05 | 5% | 13/13 | 34.1 | 11.8 | 0.4 |
| | | | 0.5 | 0.5% | 13/13 | 34.0 | 14.8 | 0.4 |
| Rxn3 | 1189M3/4 | IHEM27861 | 0.005 | 50% | 13/13 | 33.6 | 6.6 | 0.5 |
| | | | 0.05 | 10% | 13/13 | 33.8 | 9.8 | 0.5 |
| | | | 0.5 | 5% | 13/13 | 31.3 | 10.6 | 0.7 |
| | 1305M2 | IHEM27854 | 0.005 | 5% | 13/13 | 32.5 | 5.0 | 0.4 |
| | | | 0.05 | 1% | 13/13 | 31.6 | 7.6 | 0.4 |
| | | | 0.5 | 0.5% | 13/13 | 28.3 | 7.5 | 0.7 |
| | 1380M | IHEM27868 | 0.005 | 10% | 13/13 | 34.3 | 6.8 | 0.9 |
| | | | 0.05 | 5% | 13/13 | 33.3 | 9.3 | 1.0 |
| | | | 0.5 | 1% | 13/13 | 32.6 | 11.8 | 1.1 |
| Rxn4 | 1177M3 | IHEM27871 | 0.005 | 10% | 13/13 | 34.5 | 6.8 | 1.0 |
| | | | 0.05 | 1% | 13/13 | 34.6 | 10.4 | 1.0 |
| | | | 0.5 | 1% | 13/13 | 30.8 | 9.9 | 1.9 |
| | 1189M5 | IHEM27874 | 0.005 | 5% | 13/13 | 34.1 | 6.4 | 1.5 |
| | | | 0.05 | 1% | 13/13 | 32.8 | 8.6 | 1.8 |
| | | | 0.5 | 0.1% | 13/13 | 32.8 | 12.1 | 1.8 |

### B. Cross-Reactivity (Analytical Specificity)

Here, we re-tested the primers and probes for specificity by selectively performing BLAST (blastn) analysis against the other unintended viral/bacterial/fungal/human genomes. In silico analysis did not find any similarity.

In addition, we obtained genomic DNA (in ng/ul), that could be detected in the nail specimen. All assays were tested for reactivity against 1 ng genomic DNA from 54 specificity/inclusivity organisms including 13 yeast, 9 dermatophytes, 5 bacteria, 25 saprophytes and 2 controls. Exceptions was 2 controls, human genomic DNA used at 4 ng per reaction (Table 13).

TABLE 13
Cross-reactivity of TRBDR assay to bacteria, fungi and yeast

| | | TRBDR Rxn#1 | | | TRBDR Rxn#2 | | |
|---|---|---|---|---|---|---|---|
| | | TRB- | TRB- | TRB- | TRB- | TRB- | TRB- |
| Organism | Category | 1177M1 | 1189M2 | 1305M3 | 1177M2 | 1189M1 | 1305M1 |
| Candida albicans | Yeast | ND | ND | ND | ND | ND | ND |
| Candida glabarata | Yeast | ND | ND | ND | ND | ND | ND |
| Candida guillermondii | Yeast | ND | ND | ND | ND | ND | ND |
| Candida lusitaniae | Yeast | ND | ND | ND | ND | ND | ND |
| Candida parapsilosis | Yeast | ND | ND | ND | ND | ND | ND |
| Candida tropicalis | Yeast | ND | ND | ND | ND | ND | ND |
| Candida krusei | Yeast | ND | ND | ND | ND | ND | ND |
| Malassezia furfur | Yeast | ND | ND | ND | ND | ND | ND |
| Malassezia globosa | Yeast | ND | ND | ND | ND | ND | ND |
| Malassezia obtusa | Yeast | ND | ND | ND | ND | ND | ND |
| Malassezia restricta | Yeast | ND | ND | ND | ND | ND | ND |
| Malassezia sympodialis | Yeast | ND | ND | ND | ND | ND | ND |
| Epidermophyton sps | Dermatophyte | ND | ND | ND | ND | ND | ND |
| Microsporum audouinii | Dermatophyte | ND | ND | ND | ND | ND | ND |
| Microsporum canis | Dermatophyte | ND | ND | ND | ND | ND | ND |
| Microsporum gypseum | Dermatophyte | ND | ND | ND | ND | ND | ND |
| Trichophyton tonsurans | Dermatophyte | ND | ND | ND | ND | ND | ND |
| Trichophyton verr | Dermatophyte | ND | ND | ND | ND | ND | ND |
| Trichophyton violaceum | Dermatophyte | ND | ND | ND | ND | ND | ND |
| Alternaria alternata | Saprophyte | ND | ND | ND | ND | ND | ND |
| Aspergillus niger | Saprophyte | ND | ND | ND | ND | ND | ND |
| Curvularia sps | Saprophyte | ND | ND | ND | ND | ND | ND |
| Fusarium sps | Saprophyte | ND | ND | ND | ND | ND | ND |
| Scopulariopsis sps | Saprophyte | ND | ND | ND | ND | ND | ND |
| Scytyllidium | Saprophyte | ND | ND | ND | ND | ND | ND |
| Penicillium | Saprophyte | ND | ND | ND | ND | ND | ND |
| Citobacter koseri | Bacteria | ND | ND | ND | ND | ND | ND |
| Corynebacterium afermentans | Bacteria | ND | ND | ND | ND | ND | ND |
| Corynebacterium amycolatum | Bacteria | ND | ND | ND | ND | ND | ND |
| Corynebacterium minutisum | Bacteria | ND | ND | ND | ND | ND | ND |
| Corynebacterium | Bacteria | ND | ND | ND | ND | ND | ND |
| pseudotuberculosis | | | | | | | |
| Entrobacter cloacae | Bacteria | ND | ND | ND | ND | ND | ND |
| Entrobacter faecalis | Bacteria | ND | ND | ND | ND | ND | ND |
| Escherichia coli | Bacteria | ND | ND | ND | ND | ND | ND |
| Klebsiella oxytoca | Bacteria | ND | ND | ND | ND | ND | ND |
| Klebsiella pneumonia | Bacteria | ND | ND | ND | ND | ND | ND |
| Micrococcus luteus | Bacteria | ND | ND | ND | ND | ND | ND |
| Morganella morganii | Bacteria | ND | ND | ND | ND | ND | ND |
| Propionibacterium acnes | Bacteria | ND | ND | ND | ND | ND | ND |
| Proteus mirabilis | Bacteria | ND | ND | ND | ND | ND | ND |
| Pseudomonas aeruginosa | Bacteria | ND | ND | ND | ND | ND | ND |
| Pseudomonas putida | Bacteria | ND | ND | ND | ND | ND | ND |
| Serratia marcescens | Bacteria | ND | ND | ND | ND | ND | ND |
| Staphylococcus aureus | Bacteria | ND | ND | ND | ND | ND | ND |
| Staphylococcus capitis | Bacteria | ND | ND | ND | ND | ND | ND |
| Staphylococcus epidermis | Bacteria | ND | ND | ND | ND | ND | ND |
| Staphylococcus hominis | Bacteria | ND | ND | ND | ND | ND | ND |
| Staphylococcus warneri | Bacteria | ND | ND | ND | ND | ND | ND |
| Group B Staphylococcus | Bacteria | ND | ND | ND | ND | ND | ND |
| Streptococcus mitis | Bacteria | ND | ND | ND | ND | ND | ND |
| Group B Staphylococcus | Saprophyte | ND | ND | ND | ND | ND | ND |
| Bacteroides fragilis | Saprophyte | ND | ND | ND | ND | ND | ND |
| haemophilia para influenza | Virus | ND | ND | ND | ND | ND | ND |
| HPV | Virus | ND | ND | ND | ND | ND | ND |
| HSV1 | Virus | ND | ND | ND | ND | ND | ND |
| HSV2 | Virus | ND | ND | ND | ND | ND | ND |
| VZV | Virus | ND | ND | ND | ND | ND | ND |
| Scabies | Other | ND | ND | ND | ND | ND | ND |
| HugDNA | Control | ND | ND | ND | ND | ND | ND |
| ECIC DNA | Control | ND | ND | ND | ND | ND | ND |

| | | TRBDR Rxn#3 | | | TRBDR Rxn#2 4 | |
|---|---|---|---|---|---|---|
| | | TRB- | TRB- | TRB- | TRB- | TRB- |
| Organism | Category | 1189M3/4 | 1305M2 | 1380M | 1177M3 | 1189M5 |
| Candida albicans | Yeast | ND | ND | ND | ND | ND |
| Candida glabarata | Yeast | ND | ND | ND | ND | ND |
| Candida guillermondii | Yeast | ND | ND | ND | ND | ND |
| Candida lusitaniae | Yeast | ND | ND | ND | ND | ND |
| Candida parapsilosis | Yeast | ND | ND | ND | ND | ND |
| Candida tropicalis | Yeast | ND | ND | ND | ND | ND |
| Candida krusei | Yeast | ND | ND | ND | ND | ND |
| Malassezia furfur | Yeast | ND | ND | ND | ND | ND |
| Malassezia globosa | Yeast | ND | ND | ND | ND | ND |
| Malassezia obtusa | Yeast | ND | ND | ND | ND | ND |
| Malassezia restricta | Yeast | ND | ND | ND | ND | ND |
| Malassezia sympodialis | Yeast | ND | ND | ND | ND | ND |
| Epidermophyton sps | Dermatophyte | ND | ND | ND | ND | ND |
| Microsporum audouinii | Dermatophyte | ND | ND | ND | ND | ND |
| Microsporum canis | Dermatophyte | ND | ND | ND | ND | ND |
| Microsporum gypseum | Dermatophyte | ND | ND | ND | ND | ND |
| Trichophyton tonsurans | Dermatophyte | ND | ND | ND | ND | ND |
| Trichophyton verr | Dermatophyte | ND | ND | ND | ND | ND |
| Trichophyton violaceum | Dermatophyte | ND | ND | ND | ND | ND |
| Alternaria alternata | Saprophyte | ND | ND | ND | ND | ND |
| Aspergillus niger | Saprophyte | ND | ND | ND | ND | ND |
| Curvularia sps | Saprophyte | ND | ND | ND | ND | ND |
| Fusarium sps | Saprophyte | ND | ND | ND | ND | ND |
| Scopulariopsis sps | Saprophyte | ND | ND | ND | ND | ND |
| Scytyllidium | Saprophyte | ND | ND | ND | ND | ND |
| Penicillium | Saprophyte | ND | ND | ND | ND | ND |
| Citobacter koseri | Bacteria | ND | ND | ND | ND | ND |
| Corynebacterium afermentans | Bacteria | ND | ND | ND | ND | ND |
| Corynebacterium amycolatum | Bacteria | ND | ND | ND | ND | ND |
| Corynebacterium minutisum | Bacteria | ND | ND | ND | ND | ND |
| Corynebacterium | Bacteria | ND | ND | ND | ND | ND |
| pseudotuberculosis | | | | | | |
| Entrobacter cloacae | Bacteria | ND | ND | ND | ND | ND |
| Entrobacter faecalis | Bacteria | ND | ND | ND | ND | ND |
| Escherichia coli | Bacteria | ND | ND | ND | ND | ND |
| Klebsiella oxytoca | Bacteria | ND | ND | ND | ND | ND |
| Klebsiella pneumonia | Bacteria | ND | ND | ND | ND | ND |
| Micrococcus luteus | Bacteria | ND | ND | ND | ND | ND |
| Morganella morganii | Bacteria | ND | ND | ND | ND | ND |
| Propionibacterium acnes | Bacteria | ND | ND | ND | ND | ND |
| Proteus mirabilis | Bacteria | ND | ND | ND | ND | ND |
| Pseudomonas aeruginosa | Bacteria | ND | ND | ND | ND | ND |
| Pseudomonas putida | Bacteria | ND | ND | ND | ND | ND |
| Serratia marcescens | Bacteria | ND | ND | ND | ND | ND |
| Staphylococcus aureus | Bacteria | ND | ND | ND | ND | ND |
| Staphylococcus capitis | Bacteria | ND | ND | ND | ND | ND |
| Staphylococcus epidermis | Bacteria | ND | ND | ND | ND | ND |
| Staphylococcus hominis | Bacteria | ND | ND | ND | ND | ND |
| Staphylococcus warneri | Bacteria | ND | ND | ND | ND | ND |
| Group B Staphylococcus | Bacteria | ND | ND | ND | ND | ND |
| Streptococcus mitis | Bacteria | ND | ND | ND | ND | ND |
| Group B Staphylococcus | Saprophyte | ND | ND | ND | ND | ND |
| Bacteroides fragilis | Saprophyte | ND | ND | ND | ND | ND |
| haemophilia para influenza | Virus | ND | ND | ND | ND | ND |
| HPV | Virus | ND | ND | ND | ND | ND |
| HSV1 | Virus | ND | ND | ND | ND | ND |
| HSV2 | Virus | ND | ND | ND | ND | ND |
| VZV | Virus | ND | ND | ND | ND | ND |
| Scabies | Other | ND | ND | ND | ND | ND |
| HugDNA | Control | ND | ND | ND | ND | ND |
| ECIC DNA | Control | ND | ND | ND | ND | ND |

### C. Repeatability and Reproducibility:

DNA was extracted using Omega Bio-Tek's Mag-Bind Plant DNA 96 Kit on Hamilton STAR liquid handling system. The precision and accuracy of the TRBDR assay was evaluated.

Intraday/repeatability: DNA was extracted from the samples of negative nail matrix spiked with 3 levels of different percentage at & above LOD of mutant/WT mixtures at total DNA 0.05ng/rxn and were tested in 4 replicates within the same day for repeatability studies.

Interday/reproducibility: DNA was extracted from the samples of negative nail matrix spiked with 3 levels of different percentage at & above LOD of mutant/WT mixtures at total DNA at 0.05 ng/rxn in 4 replicates were tested on three different days through the TRBDR assay using different lots of reagents, independent operators, different extraction instruments, and three different PCR instruments. Results are reported as % CV of the Ct. All replicates reported are positive and within the established cut off values except where indicated. Results are shown in Tables 14 and 15. The repeatability is 9.6% CV or lower; the reproducibility is 12.5% CV or lower.

TABLE 14
TRBDR assay - Intraday/Reproducibility

| | | | | | | | | | | | |
|---|---|---|---|---|---|---|---|---|---|---|---|
| | | mutant | | | | | | | delta | delta | delta |
| | Isolate | DNA | | | Ct | Ct | | delta | Ct | Ct | Ct |
| Target | name | Percentage | CT | dRn | Mean | SD | Ct | Ct | Mean | SD | CV |

Table 14a: TRBDR reaction 1 (RXN#1):

| | | | | | | | | | | | |
|---|---|---|---|---|---|---|---|---|---|---|---|
| 1177M1 | IHEM27864 | 5% | 30.62 | 1.05 | 30.47 | 0.41 | 1.3% | 6.39 | 6.38 | 0.31 | 4.8% |
| | | | 30.22 | 1.22 | | | | 6.21 | | | |
| | | | 30.97 | 1.04 | | | | 6.82 | | | |
| | | | 30.06 | 1.46 | | | | 6.12 | | | |
| | | 10% | 29.76 | 1.60 | 29.50 | 0.22 | 0.7% | 5.95 | 5.63 | 0.28 | 4.9% |
| | | | 29.49 | 1.88 | | | | 5.45 | | | |
| | | | 29.53 | 1.87 | | | | 5.77 | | | |
| | | | 29.24 | 1.97 | | | | 5.3 | | | |
| | | 25% | 28.08 | 1.91 | 28.19 | 0.14 | 0.5% | 4.09 | 4.08 | 0.14 | 3.5% |
| | | | 28.32 | 1.83 | | | | 4.23 | | | |
| | | | 28.29 | 1.79 | | | | 4.12 | | | |
| | | | 28.06 | 1.90 | | | | 3.89 | | | |
| 1189M2 | IHEM27863 | 15% | 30.56 | 1.84 | 30.67 | 0.13 | 0.4% | 6.79 | 6.87 | 0.08 | 1.1% |
| | | | 30.70 | 1.92 | | | | 6.81 | | | |
| | | | 30.84 | 2.04 | | | | 6.90 | | | |
| | | | 30.59 | 2.16 | | | | 6.96 | | | |
| | | 25% | 28.65 | 2.37 | 28.75 | 0.08 | 0.3% | 5.17 | 5.34 | 0.12 | 2.3% |
| | | | 28.79 | 2.30 | | | | 5.35 | | | |
| | | | 28.83 | 2.39 | | | | 5.44 | | | |
| | | | 28.72 | 2.41 | | | | 5.42 | | | |
| | | 50% | 27.92 | 2.43 | 27.90 | 0.07 | 0.2% | 4.59 | 4.57 | 0.11 | 2.3% |
| | | | 27.86 | 2.48 | | | | 4.68 | | | |
| | | | 27.83 | 2.41 | | | | 4.58 | | | |
| | | | 27.98 | 2.40 | | | | 4.43 | | | |
| 1305M3 | IHEM27865 | 12.50% | 28.83 | 3.06 | 29.11 | 0.25 | 0.9% | 4.97 | 5.24 | 0.21 | 4.0% |
| | | | 29.43 | 2.42 | | | | 5.46 | | | |
| | | | 29.14 | 2.71 | | | | 5.31 | | | |
| | | | 29.02 | 3.02 | | | | 5.22 | | | |
| | | 25% | 27.73 | 2.92 | 27.59 | 0.13 | 0.5% | 4.11 | 3.93 | 0.16 | 4.0% |
| | | | 27.48 | 3.06 | | | | 3.77 | | | |
| | | | 27.48 | 3.06 | | | | 3.82 | | | |
| | | | 27.67 | 3.10 | | | | 4.01 | | | |
| | | 50% | 26.57 | 3.18 | 26.54 | 0.12 | 0.4% | 3.19 | 3.15 | 0.08 | 2.6% |
| | | | 26.50 | 3.17 | | | | 3.21 | | | |
| | | | 26.40 | 3.00 | | | | 3.03 | | | |
| | | | 26.68 | 2.86 | | | | 3.19 | | | |

Table 14b: TRBDR reaction 2 (RXN#2):

| | | | | | | | | | | | |
|---|---|---|---|---|---|---|---|---|---|---|---|
| 1177M2 | Bako-040B | 12.5% | 30.77 | 0.65 | 30.58 | 0.34 | 1.1% | 8.49 | 8.44 | 0.20 | 2.4% |
| | | | 30.75 | 0.66 | | | | 8.59 | | | |
| | | | 30.08 | 0.75 | | | | 8.15 | | | |
| | | | 30.73 | 0.70 | | | | 8.55 | | | |
| | | 25% | 28.42 | 1.08 | 28.53 | 0.07 | 0.2% | 6.47 | 6.55 | 0.08 | 1.3% |
| | | | 28.57 | 1.06 | | | | 6.48 | | | |
| | | | 28.56 | 1.08 | | | | 6.63 | | | |
| | | | 28.55 | 1.06 | | | | 6.61 | | | |
| | | 50% | 27.35 | 1.30 | 27.50 | 0.12 | 0.4% | 5.80 | 5.90 | 0.07 | 1.1% |
| | | | 27.57 | 1.19 | | | | 5.94 | | | |
| | | | 27.61 | 1.15 | | | | 5.94 | | | |
| | | | 27.47 | 1.18 | | | | 5.93 | | | |
| 1189M1 | Bako-337A | 12.5% | 32.52 | 0.51 | 33.55 | 1.36 | 4.1% | 9.80 | 10.84 | 1.35 | 12.5% |
| | | | 33.14 | 0.46 | | | | 10.60 | | | |
| | | | 35.56 | 0.38 | | | | 12.81 | | | |
| | | | 32.99 | 0.47 | | | | 10.16 | | | |
| | | 25% | 28.61 | 0.93 | 28.71 | 0.16 | 0.6% | 6.22 | 6.26 | 0.10 | 1.5% |
| | | | 28.83 | 0.90 | | | | 6.14 | | | |
| | | | 28.54 | 0.99 | | | | 6.32 | | | |
| | | | 28.86 | 0.90 | | | | 6.36 | | | |
| | | 50% | 27.07 | 1.36 | 27.19 | 0.13 | 0.5% | 4.76 | 4.74 | 0.11 | 2.2% |
| | | | 27.22 | 1.28 | | | | 4.70 | | | |
| | | | 27.13 | 1.33 | | | | 4.63 | | | |
| | | | 27.35 | 1.25 | | | | 4.88 | | | |
| 1305M1 | IHEM27860 | 10% | 29.56 | 0.84 | 29.98 | 0.55 | 1.8% | 6.92 | 7.48 | 0.66 | 8.8% |
| | | | 29.46 | 0.83 | | | | 6.95 | | | |
| | | | 30.29 | 0.74 | | | | 7.81 | | | |
| | | | 30.59 | 0.73 | | | | 8.25 | | | |
| | | 25% | 26.34 | 1.45 | 26.41 | 0.10 | 0.4% | 4.63 | 4.27 | 0.27 | 6.3% |
| | | | 26.36 | 1.44 | | | | 4.12 | | | |
| | | | 26.55 | 1.41 | | | | 4.32 | | | |
| | | | 26.40 | 1.39 | | | | 4.02 | | | |
| | | 50% | 25.15 | 1.63 | 25.27 | 0.16 | 0.6% | 3.40 | 3.41 | 0.08 | 2.3% |
| | | | 25.17 | 1.54 | | | | 3.36 | | | |
| | | | 25.50 | 1.41 | | | | 3.53 | | | |
| | | | 25.25 | 1.45 | | | | 3.36 | | | |

Table 14c: TRBDR reaction 3 (RXN#3):

| | | | | | | | | | | | |
|---|---|---|---|---|---|---|---|---|---|---|---|
| 1189M3/4 | IHEM27861 | 10% | 33.84 | 0.46 | 33.25 | 0.58 | 1.8% | 9.77 | 9.19 | 0.56 | 6.1% |
| | | | 33.63 | 0.50 | | | | 9.53 | | | |
| | | | 32.60 | 0.58 | | | | 8.55 | | | |
| | | | 32.92 | 0.51 | | | | 8.92 | | | |
| | | 25% | 29.40 | 1.01 | 29.52 | 0.09 | 0.3% | 6.32 | 6.46 | 0.16 | 2.5% |
| | | | 29.56 | 0.97 | | | | 6.42 | | | |
| | | | 29.51 | 0.99 | | | | 6.39 | | | |
| | | | 29.61 | 0.93 | | | | 6.69 | | | |
| | | 50% | 28.49 | 1.14 | 28.53 | 0.10 | 0.3% | 5.70 | 5.77 | 0.11 | 1.9% |
| | | | 28.52 | 1.10 | | | | 5.90 | | | |
| | | | 28.67 | 1.08 | | | | 5.83 | | | |
| | | | 28.44 | 1.07 | | | | 5.66 | | | |
| 1305M2 | IHEM27854 | 2.5% | 28.25 | 1.00 | 27.94 | 0.22 | 0.8% | 4.43 | 4.28 | 0.18 | 4.3% |
| | | | 27.74 | 1.06 | | | | 4.02 | | | |
| | | | 27.92 | 1.02 | | | | 4.36 | | | |
| | | | 27.85 | 1.10 | | | | 4.33 | | | |
| | | 5% | 26.23 | 1.67 | 26.46 | 0.21 | 0.8% | 3.09 | 3.30 | 0.16 | 4.9% |
| | | | 26.67 | 1.61 | | | | 3.46 | | | |
| | | | 26.35 | 1.64 | | | | 3.26 | | | |
| | | | 26.59 | 1.59 | | | | 3.39 | | | |
| | | 10% | 25.54 | 1.98 | 25.36 | 0.13 | 0.5% | 2.60 | 2.41 | 0.13 | 5.4% |
| | | | 25.27 | 1.92 | | | | 2.31 | | | |
| | | | 25.28 | 1.94 | | | | 2.35 | | | |
| | | | 25.34 | 1.85 | | | | 2.36 | | | |
| 1380M | IHEM27868 | 5% | 32.37 | 1.13 | 32.23 | 0.19 | 0.6% | 8.83 | 8.65 | 0.16 | 1.9% |
| | | | 32.39 | 1.16 | | | | 8.71 | | | |
| | | | 32.21 | 1.19 | | | | 8.57 | | | |
| | | | 31.97 | 1.25 | | | | 8.47 | | | |
| | | 10% | 30.50 | 1.68 | 30.55 | 0.20 | 0.6% | 6.81 | 6.90 | 0.30 | 4.3% |
| | | | 30.30 | 1.76 | | | | 6.53 | | | |
| | | | 30.61 | 1.66 | | | | 7.05 | | | |
| | | | 30.77 | 1.62 | | | | 7.21 | | | |
| | | 25% | 28.94 | 2.01 | 29.06 | 0.11 | 0.4% | 5.38 | 5.40 | 0.06 | 1.1% |
| | | | 29.07 | 1.98 | | | | 5.44 | | | |
| | | | 29.03 | 1.96 | | | | 5.33 | | | |
| | | | 29.20 | 1.86 | | | | 5.45 | | | |

Table 14d: TRBDR reaction 4 (RXN#4):

| | | | | | | | | | | | |
|---|---|---|---|---|---|---|---|---|---|---|---|
| 1177M3 | IHEM27871 | 5% | 31.49 | 1.91 | 31.09 | 0.34 | 1.1% | 7.33 | 6.97 | 0.31 | 4.4% |
| | | | 30.66 | 2.18 | | | | 6.59 | | | |
| | | | 31.18 | 2.15 | | | | 7.05 | | | |
| | | | 31.03 | 2.14 | | | | 6.92 | | | |
| | | 10% | 30.52 | 2.43 | 30.33 | 0.17 | 0.6% | 6.21 | 6.18 | 0.10 | 1.7% |
| | | | 30.19 | 2.63 | | | | 6.10 | | | |
| | | | 30.18 | 2.56 | | | | 6.09 | | | |
| | | | 30.42 | 2.52 | | | | 6.31 | | | |
| | | 25% | 29.01 | 2.66 | 28.93 | 0.11 | 0.4% | 4.85 | 4.82 | 0.13 | 2.7% |
| | | | 28.94 | 2.72 | | | | 4.71 | | | |
| | | | 29.00 | 2.65 | | | | 5.00 | | | |
| | | | 28.77 | 2.70 | | | | 4.73 | | | |
| 1189M5 | IHEM27874 | 1% | 31.87 | 1.76 | 31.93 | 0.24 | 0.8% | 7.52 | 7.82 | 0.33 | 4.2% |
| | | | 31.98 | 1.78 | | | | 7.89 | | | |
| | | | 31.65 | 1.85 | | | | 7.60 | | | |
| | | | 32.23 | 1.79 | | | | 8.25 | | | |
| | | 5% | 29.08 | 2.41 | 29.09 | 0.09 | 0.3% | 5.31 | 5.36 | 0.07 | 1.3% |
| | | | 28.99 | 2.44 | | | | 5.38 | | | |
| | | | 29.21 | 2.40 | | | | 5.45 | | | |
| | | | 29.06 | 2.40 | | | | 5.30 | | | |
| | | 10% | 28.65 | 2.05 | 28.66 | 0.08 | 0.3% | 5.01 | 5.03 | 0.05 | 1.0% |
| | | | 28.56 | 2.02 | | | | 4.98 | | | |
| | | | 28.74 | 1.90 | | | | 5.10 | | | |
| | | | 28.70 | 2.04 | | | | 5.04 | | | |

TABLE 15
TRBDR assay - Interday/Repeatability

| | | | | | | | | | | | |
|---|---|---|---|---|---|---|---|---|---|---|---|
| | | | | | | | | Ct | | | dCt |
| | Sample | MT/WT | Sample | Detected | Detected | Ct | Ct | CV | dCt | dCt | CV |
| Mutation | Name | (%) | # | # | % | mean | SD | % | mean | SD | % |

Table 15a: TRBDR reaction 1 (RXN#1):

| | | | | | | | | | | | |
|---|---|---|---|---|---|---|---|---|---|---|---|
| 1177M1 | IHEM27864 | 25 | 12 | 12 | 100% | 28.07 | 0.20 | 0.7% | 3.87 | 0.27 | 6.8% |
| | | 10 | 12 | 12 | 100% | 29.52 | 0.25 | 0.9% | 5.59 | 0.25 | 4.5% |
| | | 5 | 12 | 12 | 100% | 30.54 | 0.50 | 1.6% | 6.57 | 0.43 | 6.5% |
| 1189M2 | IHEM27863 | 50 | 12 | 12 | 100% | 27.87 | 0.12 | 0.4% | 4.71 | 0.15 | 3.3% |
| | | 25 | 12 | 12 | 100% | 28.77 | 0.06 | 0.2% | 5.36 | 0.15 | 2.8% |
| | | 15 | 12 | 12 | 100% | 30.63 | 0.14 | 0.5% | 6.81 | 0.14 | 2.0% |
| 1305M3 | IHEM27865 | 50 | 12 | 12 | 100% | 26.39 | 0.22 | 0.8% | 2.93 | 0.20 | 6.8% |
| | | 25 | 12 | 12 | 100% | 27.46 | 0.17 | 0.6% | 3.81 | 0.17 | 4.5% |
| | | 12.5 | 12 | 12 | 100% | 29.27 | 0.25 | 0.9% | 5.34 | 0.23 | 4.3% |

Table 15b: TRBDR reaction 2 (RXN#2):

| | | | | | | | | | | | |
|---|---|---|---|---|---|---|---|---|---|---|---|
| 1177M2 | Bako-040B | 50 | 12 | 12 | 100% | 27.79 | 0.25 | 0.9% | 6.13 | 0.21 | 3.4% |
| | | 25 | 12 | 12 | 100% | 28.85 | 0.25 | 0.9% | 6.85 | 0.27 | 4.0% |
| | | 12.5 | 12 | 12 | 100% | 31.32 | 0.71 | 2.3% | 9.10 | 0.70 | 7.7% |
| 1189M1 | Bako-337A | 50 | 12 | 12 | 100% | 27.18 | 0.14 | 0.5% | 4.72 | 0.11 | 2.3% |
| | | 25 | 12 | 12 | 100% | 28.53 | 0.19 | 0.6% | 6.14 | 0.22 | 3.6% |
| 1305M1 | IHEM27860 | 50 | 12 | 12 | 100% | 25.13 | 0.32 | 1.3% | 3.25 | 0.25 | 7.6% |
| | | 2.5 | 12 | 12 | 100% | 26.23 | 0.31 | 1.2% | 4.17 | 0.21 | 5.0% |
| | | 10 | 12 | 12 | 100% | 29.85 | 0.70 | 2.3% | 7.39 | 0.71 | 9.6% |

Table 15c: TRBDR reaction 3 (RXN#3):

| | | | | | | | | | | | |
|---|---|---|---|---|---|---|---|---|---|---|---|
| 1189M3/4 | IHEM27861 | 50 | 12 | 12 | 100% | 28.51 | 0.10 | 0.3% | 5.72 | 0.16 | 2.7% |
| | | 25 | 12 | 12 | 100% | 29.57 | 0.13 | 0.5% | 6.46 | 0.14 | 2.1% |
| | | 10 | 12 | 12 | 100% | 32.91 | 0.59 | 1.8% | 9.11 | 0.47 | 5.2% |
| 1305M2 | IHEM27854 | 10 | 12 | 12 | 100% | 25.52 | 0.21 | 0.8% | 2.46 | 0.16 | 6.6% |
| | | 5 | 12 | 12 | 100% | 26.54 | 0.18 | 0.7% | 3.29 | 0.22 | 6.6% |
| | | 2.5 | 12 | 12 | 100% | 27.84 | 0.24 | 0.8% | 4.23 | 0.22 | 5.1% |
| 1380M | IHEM27868 | 25 | 12 | 12 | 100% | 29.04 | 0.16 | 0.6% | 5.49 | 0.17 | 3.1% |
| | | 10 | 12 | 12 | 100% | 30.58 | 0.23 | 0.8% | 7.04 | 0.32 | 4.6% |
| | | 5 | 12 | 12 | 100% | 32.16 | 0.28 | 0.9% | 8.44 | 0.28 | 3.4% |

Table 15d: TRBDR reaction 4 (RXN#4):

| | | | | | | | | | | | |
|---|---|---|---|---|---|---|---|---|---|---|---|
| 1177M3 | IHEM27871 | 25 | 12 | 12 | 100% | 29.23 | 0.27 | 0.9% | 5.01 | 0.25 | 5.0% |
| | | 10 | 12 | 12 | 100% | 30.53 | 0.26 | 0.8% | 6.37 | 0.22 | 3.5% |
| | | 5 | 12 | 12 | 100% | 31.44 | 0.39 | 1.2% | 7.30 | 0.38 | 5.2% |
| 1189M5 | IHEM27874 | 10 | 12 | 12 | 100% | 28.30 | 0.30 | 1.1% | 4.74 | 0.23 | 5.0% |
| | | 5 | 12 | 12 | 100% | 29.21 | 0.16 | 0.6% | 5.45 | 0.16 | 2.9% |
| | | 1 | 12 | 12 | 100% | 32.16 | 0.31 | 1.0% | 8.01 | 0.31 | 3.9% |

### D. Analytical Interference:

The endogenous control target in the assay serves as control for the interference of the specimen for effective nucleic acid isolation, preparation, or extraction and target amplification. The amplification of endogenous control target can mitigate the risk of interference exists in the specimen. Also the PCR positive control (TRBDR-Pos Ctrls), ensures PCR reagents are performing as intended and is used on every plate at a concentration.

### E. Reagent Stability:

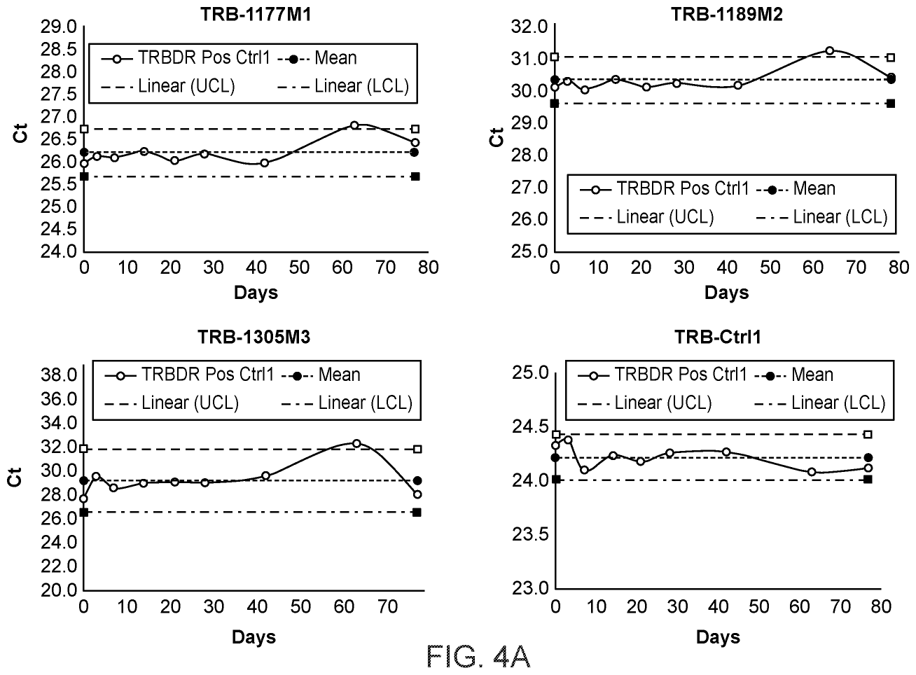

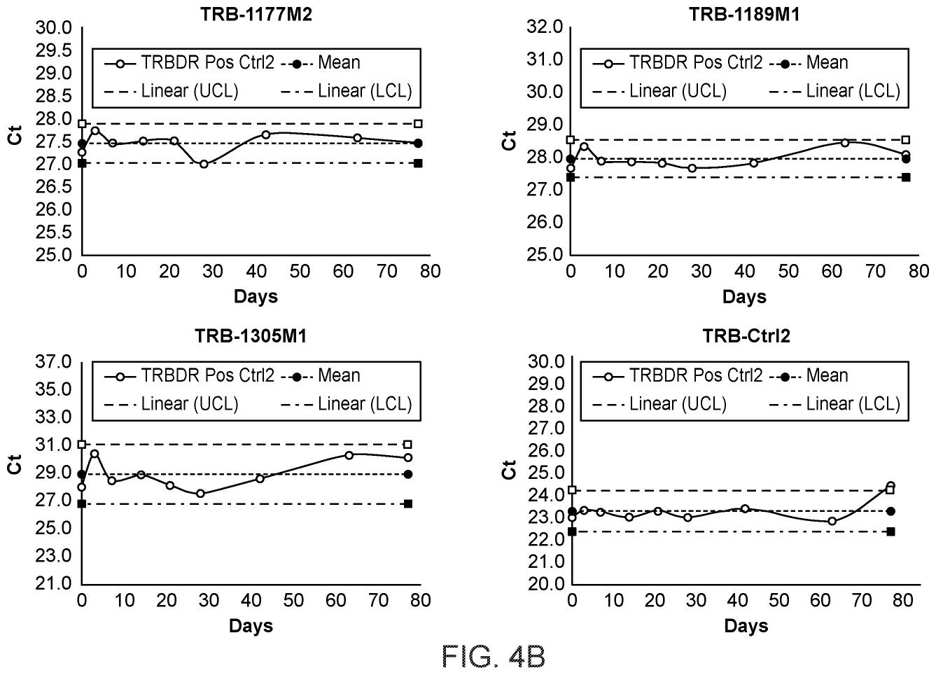

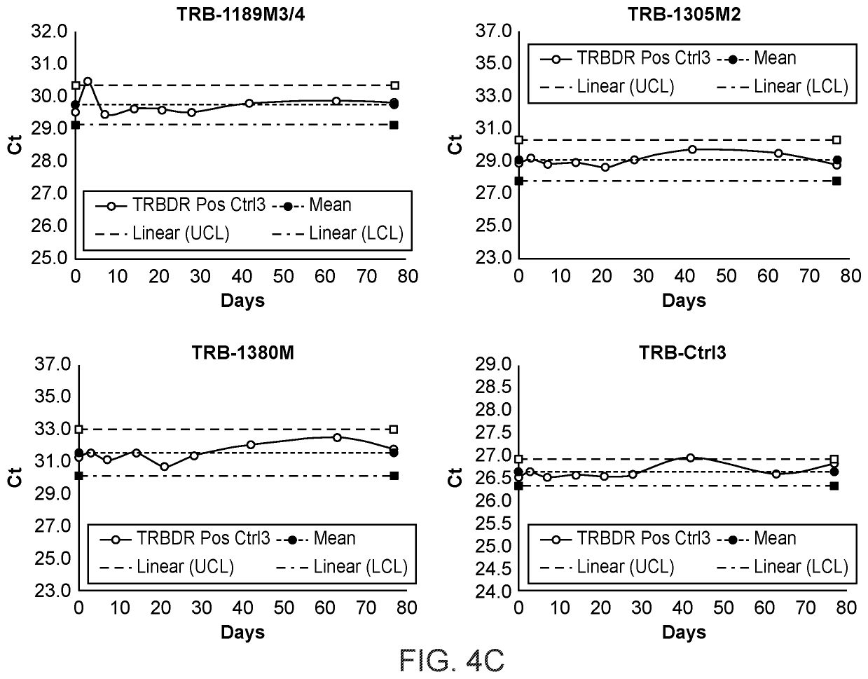

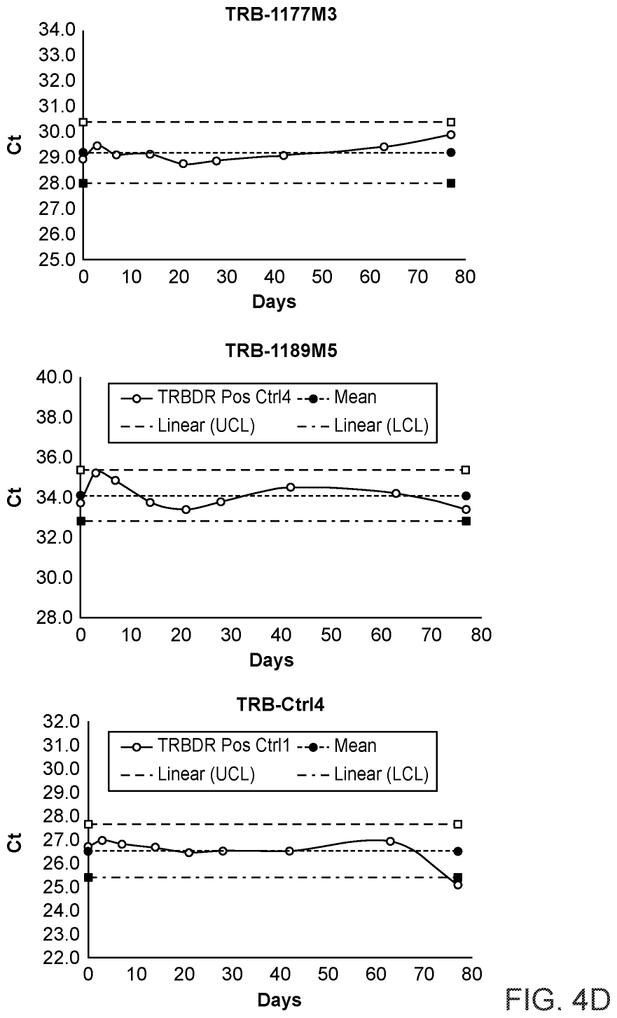

The positive control plasmid containing the template for the all the mutation and endogenous control sequence targets for each reactions was designed and synthesized from GenScript USA. Four plasmids for four reactions were used. The plasmids were diluted in DBS buffer, the concentration is determined by NanoDrop and diluted to the working concentration to be used to test the PCR assay reagent stability continuously for 77 days real time of days 0, 3, 7, 14, 21, 28, 42, 63 and 77 which represent the longest time for each preparation of the reagent lots of 50× Primer/Probe mixture and 3.5× enzyme mastermix. The results of the reagent stability for the TRBDR assay for all the targets results was shown as a Levey-Jennings plot FIG. 4. The plot shows the target mean Ct value (solid blue line) as well as expected limits (grey hatched lines) (two standard deviations). All control results are within two standard deviations of the mean target value.

### F. Laboratory Evaluation:

These approaches were used for the validation of the TRBDR Assay.

1. Laboratory evaluation with contrived clinical specimens

2. Culture correlation to TRBDR Assay using drug resistance clinical samples or isolates.

3. Correlation of TRBDR Assay to another molecular method with different methodology

1. Contrived Sample Testing:
A contrived clinical study was performed to evaluate the performance of TRBDR Assay using 22 terbinafine drug resistance isolates from BCCM. A total of 363 samples of different mutant isolates, at percentages near LOD of mutant/WT mixtures at different total DNA level were spiked into the clinical negative nail matrix and DNA was extracted and tested across all the four reactions of the TRBDR Assay. Thirty Three contrived samples were tested for each target and the rest 330 samples serves as negative samples for the same target. The Table 16 summarizes the clinical performance of the TRBDR Assay using contrived specimens.

TABLE 16
Clinical evaluation of TRBDR Assay on
contrived positive and negative samples

| | | | | |
|---|---|---|---|---|
| Mutation | | | | # Not |
| (Isolate) | Spike-In Levels | Samples | # Detected | Detected |

Table 16a: TRBDR reaction 1 (RXN#1):

| | | | | |
|---|---|---|---|---|
| 1177-M1 | 0.5 ng - 5% | 11 | 11 | 0 |
| (IHEM27864) | 0.05 ng - 5% | 11 | 10 | 1 |
| | 0.005 ng - 50% | 11 | 11 | 0 |
| | Negative | 330 | 0 | 330 |
| 1189-M2 | 0.5 ng - 5% | 11 | 11 | 0 |
| (IHEM27863) | 0.05 ng - 25% | 11 | 11 | 0 |
| | 0.005 ng - 100% | 11 | 11 | 0 |
| | Negative | 330 | 0 | 330 |
| 1305-M3 | 0.5 ng - 1% | 11 | 11 | 0 |
| (IHEM27865) | 0.05 ng - 5% | 11 | 11 | 0 |
| | 0.005 ng - 50% | 11 | 11 | 0 |
| | Negative | 330 | 0 | 330 |

Table 16b: TRBDR reaction 2 (RXN#2):

| | | | | |
|---|---|---|---|---|
| 1177-M2 (040B) | 0.5 ng - 5% | 11 | 11 | 0 |
| | 0.05 ng - 10% | 11 | 11 | 0 |
| | 0.005 ng - 100% | 11 | 11 | 0 |
| | Negative | 330 | 0 | 330 |
| 1189-M1 (337A) | 0.5 ng - 5% | 11 | 11 | 0 |
| | 0.05 ng - 10% | 11 | 11 | 0 |
| | 0.005 ng - 100% | 11 | 10 | 1 |
| | Negative | 330 | 0 | 330 |
| 1305-M1 | 0.5 ng - 1% | 11 | 11 | 0 |
| (IHEM27860) | 0.05 ng - 10% | 11 | 11 | 0 |
| | 0.005 ng - 100% | 11 | 11 | 0 |
| | Negative | 330 | 0 | 330 |

Table 16c: TRBDR reaction 3 (RXN#3):

| | | | | |
|---|---|---|---|---|
| 1189-M3/4 | 0.5 ng - 5% | 11 | 11 | 0 |
| (IHEM27861) | 0.05 ng - 25% | 11 | 11 | 0 |
| | 0.005 ng - 100% | 11 | 11 | 0 |
| | Negative | 330 | 0 | 330 |
| 1305-M2 | 0.5 ng - 1% | 11 | 11 | 0 |
| (IHEM27854) | 0.05 ng - 5% | 11 | 11 | 0 |
| | 0.005 ng - 25% | 11 | 11 | 0 |
| | Negative | 330 | 0 | 330 |
| 1380-M | 0.5 ng - 5% | 11 | 11 | 0 |
| (IHEM27868) | 0.05 ng - 10% | 11 | 11 | 0 |
| | 0.005 ng - 50% | 11 | 11 | 0 |
| | Negative | 330 | 0 | 330 |

Table 16d: TRBDR reaction 4 (RXN#4):

| | | | | |
|---|---|---|---|---|
| 1177-M3 | 0.5 ng - 5% | 11 | 11 | 0 |
| (IHEM27871) | 0.05 ng - 10% | 11 | 11 | 0 |
| | 0.005 ng - 50% | 11 | 11 | 0 |
| | Negative | 330 | 0 | 330 |
| 1189-M5 | 0.5 ng - 10% | 11 | 11 | 0 |
| (IHEM27874) | 0.05 ng - 10% | 11 | 11 | 0 |
| | 0.005 ng - 50% | 11 | 11 | 0 |
| | Negative | 330 | 0 | 330 |

Performance of the TRBDR Assay against the expected results were:

Positive percent agreement: 361/363=99.4%

Negative percent agreement: 363/363=100%

2. Culture Correlation to TRBDR Assay Using Drug Resistance Clinical Samples or Isolates:

Two hundred fifty-nine clinical nail specimens (Tinea unguium) and 22 terbinafine drug resistance isolates from BCCM were placed on the PDA plate, for fungal culture and detection. Culture positive samples further identified by microscopy and VITEK® MS (Biomerieux) as Trichophyton sps, were further cultured on SDA plate containing 0.06 g/l terbinafine, 0.4 g/l of cycloheximide and 0.05 g/l of chloramphenicol, for initial screening their terbinafine resistance and re-cultured on fresh SDA plate containing 0.06 g/l terbinafine. Thirteen were culture positive on fresh terbinafine SDA plate. DNA was extracted from terbinafine susceptible and resistant fungal cultures using Omega Bio-Tek's Mag-Bind Plant DNA 96 Kit on Hamilton STAR liquid handling system. The Extracted DNA from the isolates is tested with TRBDR assay. The results of this comparison studies are shown in Tables 17.

TABLE 17
Clinical validation of TRBDR assay vs fungal culture for terbinafine
drug resistance using clinical samples and isolates

Culture terbinafine resistance

| | | Positive | Negative | Total |
|---|---|---|---|---|
| TRBDR | Positive | 35 | 0 | 35 |
| Assay | Negative | 0 | 246 | 246 |
| | Total | 35 | 246 | 281 |

Performance of the TRBDR assay against the fungal culture results were:

Sensitivity: 35/35=100%

Specificity: 246/246=100%

Moreover, full length squalene epoxidase (SQLE) gene Sanger sequencing was performed for all the terbinafine culture positive clinical samples and isolates. Drug resistance mutations were detected in all the 35 samples, which are also positive in the TRBDR assay.

3. Correlation of TRBDR Assay to Another Molecular Method with Different Methodology:

There is no regulatory approved molecular method for the terbinafine resistance mutation detection for TRBDR assay panel validation. Moreover, Sanger sequencing can not differentiate mutant versus wild type allele in mixed infections where both mutant and wild type coexist. Thus an alternate comparable molecular method was developed to detect the terbinafine drug resistance mutations in squalene epoxidase gene based on the Proofreading PCR methodology described in Hao et al 2015 PloS One DOI:10.1371/journal.pone.0123468. Proofreading PCR (PR-PCR) method using a ddNTP blocked primer for the wildtype allele extension at the 3′ end and a mixture of DNA polymerases with and without the 3′-5′ proofreading function. The ddNTP blocked primer exhibited the best blocking efficiency to avoid nonspecific primer extension while the mixture of a tiny amount of high-fidelity DNA polymerase with a routine amount of Taq DNA polymerase provided the best discrimination and amplification effects. The PR-PCR method is quite capable of detecting point mutations and allows discrimination amplification when the mismatch is located within the last eight nucleotides from the 3′-end of the ddNTP blocked primer.

PR-PCR applications were performed using the following primers:

TABLE 18
list of primers used for mutation
detection for squalene epoxidase
gene in PR-PCR

| | | | SEQ |
|---|---|---|---|
| | | | ID |
| Mutation | Primer | Sequence | NOs |
| 1177M1-3 | 1177M-PR-F | CATCAATATTCTTGCTCAGGCCT | 55 |
| | | TATAC* | |
| | 1177M-PR-R | TGCAGGACTTTCAAATTRGGGT | 56 |
| 1189M1-5 | 1189M-PR-F | TCTTGCTCAGGCCTTATACTCTA | 57 |
| | | TATTC* | |
| | 1189M-PR-R | TGCAGGACTTTCAAATTRGGGT | 58 |
| 1305M1-3 | 1305M-PR-F | ACGAGGCTGCTTCAGGTATTTC* | 59 |
| | 1305M-PR-R | TGTCTAGCTTTGAAGTTCGGCAA | 60 |
| 1380M | 1380M-PR-F | TCACCCTTGGTTCTATTACGGC* | 61 |
| | 1380M-PR-R | TGTCTAGCTTTGAAGTTCGGCAA | 62 |

*with 3′-terminal nucleotide with ddCTP blocker

TABLE 19
List of primers used for NEST-PR-PCR
for mutafter the PR-PCR ation detection
for squalene epoxidase gene

| | | | SEQ |
|---|---|---|---|
| | | | ID |
| Mutation | Primer | Sequence | NOs |
| 1177M1/3 | 1177M13- | ATCAATATTCTTGCYCAGKCCT | 63 |
| | NEST-F | TY | |
| | 1177M13- | TGCAGGACTTTCAAATTRGGGT | 64 |
| | NEST-R | | |
| 1177M2 | 1177M2- | ATCAATATTCTTGCTCAGGCCT | 65 |
| | NEST-R | C | |
| | 1177M2- | TGCAGGACTTTCAAATTRGGGT | 66 |
| | NEST-F | | |
| 1189M1/5 | 1189M15- | CTTGCTCAGGCCTTATACTCTA | 67 |
| | NEST-F | TATTR | |
| | 1189M15- | TGCAGGACTTTCAAATTRGGGT | 68 |
| | NEST-R | | |
| 1189M2/3/4 | 1189M234- | CTTGCTCAGGCCTTATACTCTA | 69 |
| | NEST-F | TAV | |
| | 1189M234- | TGCAGGACTTTCAAATTRGGGT | 70 |
| | NEST-R | | |
| 1305M1/2 | 1305M12- | ACGAGGCTGCTTCAGGTATR | 71 |
| | NEST-F | | |
| | 1305M12- | AGATGGGTTTGCTAGTAAGGTG | 72 |
| | NEST-R | TAG | |
| 1305M3 | 1305M3- | CGAGGCTGCTTCAGGTATTC | 73 |
| | NEST-F | | |
| | 1305M3- | AGATGGGTTTGCTAGTAAGGTG | 74 |
| | NEST-R | TAG | |
| 1380M | 1380M- | CACCCTTGGTTCTATTACGRT | 75 |
| | NEST-F | | |
| | 1380M- | TGTCTAGCTTTGAAGTTCGGCA | 76 |
| | NEST-R | A | |

To establish the PR-PCR methodology for the detection of target mutation for drug resistance, wild type, mutant and mixtures of mutant/wildtype at different percentages ranging from 100% to 0.1% DNA was tested. PR-PCR is specific to detect only the mutant allele, no amplification was observed with the wild type allele by agarose gel electrophoresis, which is confirmed by Sanger sequencing. Sanger sequencing results of PCR products of SQLE gene without PR-PCR, can only detect wild type and mutant allele at 100%, but cannot differentiate the wild type and mutant allele in the mutant & wildtype mixtures containing 50 −1% mutant. However Sanger sequencing results of the PR-PCR products can detect the mutant in the mixture as low as 1% for 1189M1 and 1177M1-3 mutation as an example. PR-PCR for all the other mutations were also confirmed by Sanger Sequencing.

1301 clinical nail samples which are confirmed to be Trichophyton species from OIAD Dermatophytic Fungi Reflex Assay, of which 87.2% are T. rubrum and 12.8% are T. mentagrophytes, were tested with both TRBDR Assay panel for the presence of Terbinafine 15 resistance mutations. Among the 1301 clinical samples tested, all the terbinafine resistant mutation positive and 250 negative samples were further tested and compared with the above Sanger sequencing-confirmed PR-PCR methodology.

The correlation between the results of TRBDR Assay panel and PR-PCR were analyzed and shown in Table 20 and 21.

TABLE 20
Clinical correlation of TRBDR assay vs PR-PCR
methodology for terbinafine drug resistant
mutation detection using clinical samples

PR-PCR

| | | Positive | Negative | Total |
|---|---|---|---|---|
| TRBDR | Positive | 51 | 14 | 65 |
| Assay | Negative | 5 | 250 | 255 |
| | Total | 56 | 264 | 320 |

Performance of the TRBDR assay against the Sanger sequencing confirmed PR-PCR results were:

Sensitivity: 51/56=91%

Specificity: 250/264=95%

TABLE 21
Clinical correlation of TRBDR assay based on individual terbinafine
drug resistant mutation comparing with PR-PCR methodology

| | | Comparing with PR-PCR | | | | |
|---|---|---|---|---|---|---|
| | | | | TRBDR | PR- | |
| | | | | Assay | PCR | |
| | mutation | Both | Both | positive | positive | |
| | target | positive | negative | only | only | Total |
| TRBDR | TRB-1177M1 | 1 | 75 | 0 | 0 | 76 |
| Assay | TRB-1177M2 | 0 | 114 | 0 | 2 | 116 |
| | TRB-1177M3 | 2 | 75 | 0 | 0 | 77 |
| | TRB-1189M1 | 9 | 86 | 4 | 0 | 99 |
| | TRB-1189M2 | 21 | 82 | 6 | 1 | 110 |
| | TRB-1189M3/ | 3 | 82 | 4 | 0 | 89 |
| | M4 | | | | | |
| | TRB-1189M5 | 1 | 86 | 0 | 0 | 87 |
| | TRB-1305M1 | 1 | 61 | 0 | 0 | 62 |
| | TRB-1305M2 | 1 | 61 | 0 | 0 | 62 |
| | TRB-1305M3 | 4 | 51 | 0 | 0 | 55 |
| | TRB-1380M | 8 | 63 | 0 | 2 | 73 |
| Total | | 51 | 250 | 14 | 5 | 320 |

### REFERENCES

1. Gupta A K, et al., 2020. The Growing Problem of Antifungal Resistance in Onychomycosis and Other Superficial Mycoses. Am J Clin Dermatol. 2020 Dec 22. doi: 10.1007/s40257-020-00580-6. Online ahead of print.

2. Fattahi A, et al., 2020. Multidrug-resistant Trichophyton mentagrophytes genotype VIII in an Iranian family with generalized dermatophytosis: report of four cases and review of literature. Int J Dermatol. 2020 Oct 13. doi: 10.1111/ijd.15226. Online ahead of print.

3. Saunte DML, et al., 2019. Emerging terbinafine resistance in Trichophyton: clinical characteristics, squalene epoxidase gene mutations and a reliable EUCAST method for detection. Antimicrob Agents Chemother. [Epub ahead of print] PubMed PMID: 31383665.

4. Singh A, et al., 2018. High terbinafine resistance in Trichophyton interdigitale isolates in Delhi, India harbouring mutations in the squalene epoxidase gene. Mycoses. 61(7):477-484. PubMed PMID: 29577447.

5. Yamada T, et al., 2017. Terbinafine Resistance of Trichophyton Clinical Isolates Caused by Specific Point Mutations in the Squalene Epoxidase Gene. Antimicrob Agents Chemother. 61(7). pii: e00115-17. PubMed PMID: 28416557

6. Ghelardi E, et al., 2014. Potential of Ergosterol synthesis inhibitors to cause resistance or cross-resistance in Trichophyton rubrum. Antimicrob Agents Chemother. 58(5):2825-9. PubMed PMID: 24614379

7. Nowosielski M, et al., 2011. Detailed mechanism of squalene epoxidase inhibition by terbinafine. J Chem Inf Model. 51(2):455-62. PubMed PMID: 21229992.

8. Martinez-Rossi N M, et al., 2018. Dermatophyte Resistance to Antifungal Drugs: Mechanisms and Prospectus. Front. Microbiol. 9:1108. doi: 10.3389/fmicb.2018.01108

9. Hao W, et al., 2015. Modified Proofreading PCR for Detection of Point Mutations, Insertions and Deletions Using a ddNTP-Blocked Primer. PLoS ONE 10(4): e0123468. PubMed PMID: 25915410

What is claimed is:

1. A method of sample analysis comprising:
(a) hybridizing a tailed primer to a sample that comprises both wild type copies of a genomic locus and mutant copies of the genomic locus that have a point mutation relative to the wild type copies of the genomic locus,
wherein the tailed primer comprises:
i. a target complementary sequence that is fully complementary to a target site in the mutant locus and includes a 3′ terminal nucleotide that base pairs with the point mutation; and
ii. a 5′ tail that is not complementary to the genomic locus;
(b) extending the tailed primer using the genomic locus as a template to produce a primer extension product; and
(c) detecting the primer extension product in a quantitative polymerase chain reaction (qPCR) assay that employs:
(i) a forward primer that is complementary to a sequence in the complement of the 5′ tail of the tailed primer;
(ii) a reverse primer that hybridizes to a site in the genomic locus on the opposite strand and downstream from the site to which the tailed primer binds;
(iii) a hydrolysis probe that is complementary to:
at least 6 nucleotides of the target complementary sequence of the tailed primer; and
at least 6 nucleotides of the 5′ tail of the tailed primer.

2. The method of claim 1, wherein the method comprises combining the tailed primer, sample, forward primer, reverse primer, and hydrolysis probe with polymerase and nucleotides in a reaction vessel to produce a reaction mixture and thermocycling the reaction mixture without opening the reaction vessel or adding additional reagents to the reaction mixture after step (a) and before step (c).

3. The method of claim 1, wherein:
i. the Tₘ of the target complementary sequence of the tailed primer is at least 8° C. lower than the Tₘs of the forward primer, the reverse primer and the hydrolysis probe; and
ii. the method comprises subjecting the reaction mixture to the following thermocycling conditions:
a first set of 1-5 cycles that comprise a denaturation step followed by a first incubation at a temperature in the range of 40° C. to 52° C., a second incubation step at a temperature in the range of 55° C. to 65° C., a third incubation step at a temperature in the range of 65° C. to 75° C.; and
a second set of 20-50 cycles that comprise a denaturation step followed by an incubation at one or more temperatures that are at least 8° C. higher than the temperature of the first incubation in the first set of cycles.

4. The method of claim 1, wherein:
i. the Tₘ of the target complementary sequence of the tailed primer is less than 52° C. and the Tₘs of the forward primer, the reverse primer and the hydrolysis probe are at least 60° C.; and
ii. the method comprises subjecting the reaction mixture to the following thermocycling conditions:
a first set of 1-5 cycles of a first temperature of at least 90° C., a second temperature in the range of 40° C. to 52° C., a third temperature in the range of 55° C. to 65 and a fourth temperature in the range of 65° C. to 75° C.; followed by:
a second set of 20-50 cycles of a fifth temperature of at least 90° C., a sixth temperature that is at least 8° C. higher than the second temperature, and an optional seventh temperature in the range of 65° C. to 75° C.

5. The method of claim 1, wherein the target complementary sequence of the tailed primer is in the range of 11-26 nucleotides and the forward primer, reverse primer and hydrolysis probes are at least 25 nucleotides in length.

6. The method of claim 1, wherein the sample is obtained from a human.

7. The method of claim 1, wherein the sample is obtained from a human and the tailed primer and reverse primer are complementary to sequences in a pathogen.

8. The method of claim 1, wherein the point mutation is responsible for antibiotic resistance.

9. The method of claim 7, wherein the pathogen is a fungal pathogen.

10. The method of claim 1, wherein the sample contains at least 100 times more wild type copies of the genomic locus than mutant copies of the genomic locus.

11. A reaction mixture comprising:
(a) a sample that comprises both wild type copies of a genomic locus and mutant copies of the genomic locus that have a point mutation relative to the wild type copies of the genomic locus;
(b) a tailed primer, wherein the tailed primer comprises:
i. a target complementary sequence that is fully complementary to a target site in the genomic locus and includes a 3′ terminal nucleotide that base pairs with the point mutation in the locus; and
ii. a 5′ tail that is not complementary to the genomic locus;
(c) a forward primer that is complementary to a sequence in the complement of the 5′ tail of the tailed primer;
(d) a reverse primer that hybridizes to a site in the genomic locus on the opposite strand and downstream from the site to which the tailed primer binds;
(e) a hydrolysis probe that is complementary to:
at least 6 nucleotides of the target complementary sequence of the tailed primer; and
at least 6 nucleotides of the 5′ tail of the tailed primer.

12. The reaction mixture of claim 11, further comprising a polymerase and dNTPs.

13. The reaction mixture of claim 11, wherein the target complementary sequence of the tailed primer is in the range of 11-26 nucleotides and the forward primer, reverse primer and hydrolysis probes are at least 25 nucleotides in length.

14. A kit comprising:
(a) a tailed primer, wherein the tailed primer comprises:
iii. a target complementary sequence that is fully complementary to a target site in a genomic locus and includes a 3′ terminal nucleotide that base pairs with a point mutation in the locus; and
iv. a 5′ tail that is not complementary to the genomic locus;
(b) a forward primer that is complementary to a sequence in the complement of the 5′ tail of the tailed primer;
(c) a reverse primer that hybridizes to a site in the genomic locus on the opposite strand and downstream from the site to which the tailed primer binds;
(d) a hydrolysis probe that is complementary to:
at least 6 nucleotides of the target complementary sequence of the tailed primer; and
at least 6 nucleotides of the 5′ tail of the tailed primer.

15. The kit of claim 14, wherein the target complementary sequence of the tailed primer is in the range of 11-26 nucleotides and the forward primer, reverse primer and hydrolysis probes are at least 25 nucleotides in length.
